(19) United States
(12) Patent Application Publication
(10) Pub. No.: US 2023/0112085 A1
(43) Pub. Date: Apr. 13, 2023

(54) ANTI-B7-H4 CONSTRUCTS AND USES THEREOF

(71) Applicant: Apeximmune Therapeutics Inc., South San Francisco, CA (US)

(72) Inventors: Li-Fen LEE, South San Francisco, CA (US); Kan LU, South San Francisco, CA (US); Jessica YU, South San Francisco, CA (US); Sheng-Tien LI, Taipei City (TW); Julie HUANG, South San Francisco, CA (US)

(21) Appl. No.: 17/796,152

(22) PCT Filed: Jan. 29, 2021
(86) PCT No.: PCT/US2021/015925

§ 371 Date: Jul. 28, 2022

Related U.S. Application Data
(60) Provisional application No. 62/968,070, filed on Jan. 30, 2020.

(51) Int. Cl. C07K 16/28; A61P 35/00

(52) U.S. Cl. CPC C07K 16/2827; C07K 16/2818; A61P 35/00

(57) ABSTRACT

The present application provides anti-B7-H4 constructs that bind to B7-H4 (e.g., anti-B7-H4 antibodies), nucleic acid molecules encoding an amino acid sequence of the anti-B7-H4, vectors comprising the nucleic acid molecules, host cells containing the vectors, methods of preparing the anti-B7-H4 construct, pharmaceutical compositions containing the anti-B7-H4 construct, and methods of using the anti-B7-H4 construct or compositions.

### CROSS REFERENCE TO RELATED APPLICATIONS

This application claims priority benefit of U.S. Provisional Application 62/968,070 filed Jan. 30, 2020, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 196882000240SEQLIST.TXT, date recorded: Jan. 29, 2021, size: 30 KB).

### TECHNICAL FIELD

The present disclosure relates to anti-B7-H4 constructs (such as anti-B7-H4 antibodies) and the uses thereof.

### BACKGROUND OF THE APPLICATION

B7-H4 or B7x/B7s is a member of the B7 superfamily and has recently identified as an inhibitory modulator of T-cell response (Sica et al., 2003, Immunity 18(6):849-61; Prasad et al., 2003, Immunity 18(6):863-73; Zang et al., 2003, Proc Natl Acad Sci USA, 100(18):10388-92). When present at the surface of antigen presenting cells, B7-H4 negatively regulates T cell activation, possibly through interaction with a ligand that remains to be identified (Kryczek et al., 2006, J Immunol 177(1):40-4). B7-H4 adenoviral overexpression in pancreatic islets protected mice from autoimmune diabetes maintaining peripheral tolerance (Wei et al., 2011, J Exp Med, 208(8):1683-94). Consistently with this observation, B7-H4 knock-out mice are more resistant to infection by Listeria monocytogenes than their wild type littermates due to a higher proliferation of neutrophils in peripheral organs (Zhu et al., 2009, Blood, 113(8):1759-67).

B7-H4 is widely expressed at the mRNA level, but its restricted pattern of protein expression in normal tissues suggests posttranscriptional regulation. B7-H4 expression in tumor tissues was observed in various types of human cancers such as breast (Tringler et al., 2005, Clin Cancer Res 11(5):1842-8), ovarian (Kryczek et al., 2006, J Exp Med 203(4):871-81), pancreatic, lung (Choi et al., 2003, J Immunol 171(9):4650-4; Sun et al., 2006, Lung Cancer 53(2):143-51) melanoma (Quandt et al., 2011, Clin Cancer Res 17(10):3100-11) and renal cell carcinoma (Jung et al., 2011, Korean J Urol 52(2):90-5; Krambeck et al., 2006. Proc Natl Acad Sci USA 103(27):10391-6). B7-H4 expression was evaluated by immunohistochemistry in most studies, either as a cytoplasm or a plasma membrane protein (Quandt et al., 2011, Clin Cancer Res 17(10):3100-11; Krambeck et al., 2006, Proc Natl Acad Sci USA 103(27):10391-6; Jiang et al., 2010, Cancer Immunol Immunother 59(11):1707-14; Zang et al., 2007. Proc Natl Acad Sci USA, 104(49):19458-63; Miyatake et al., 2007, Gynecol Oncol 106(1):119-27). In ovarian cancer cells, B7-H4 expression was assessed by flow cytometry and was also reported to be mainly intracellular (Kryczek et al., 2006, J Exp Med 203(4):871-81), to the exception of some cell lines where cell surface expression was observed (Choi et al., 2003, J Immunol 171(9):4650-4). B7-H4 has also been detected in a soluble form in blood samples from cancer patients (Simon et al., 2006, Cancer Res 66(3):1570-5; Thompson et al., 2008, Cancer Res 68(15):6054-8). The broad presence in various cancers of a negative regulator of T cell activation suggests a role of B7-H4 in down-regulating antitumor immunity.

The disclosures of all publications, patents, patent applications and published patent applications referred to herein are hereby incorporated herein by reference in their entirety.

### BRIEF SUMMARY OF THE APPLICATION

The following summary is illustrative only and is not intended to be limiting in any way. That is, the following summary is provided to introduce highlights, benefits and advantages of the novel molecules and the uses thereof. Thus, the following summary is not intended to identify essential features of the claimed subject matter, nor is it intended for use in determining the scope of the claimed subject matter.

The present application provides anti-B7-H4 constructs and uses thereof.

In one aspect of the present application, there is provided an anti-B7-H4 construct comprising an antibody moiety comprising a heavy chain variable region (V_{H}) and a light chain variable region (V_{L}), wherein the antibody moiety competes for a binding epitope of B7-H4 with an antibody or antibody fragment comprising a second heavy variable region (V_{H-2}) and a second light chain variable region (V_{L-2}), wherein: a) the V_{H-2} comprises the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L}.2 comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6; b) the V_{H-2} comprises the HC-CDR1 comprising the amino acid sequence of SEQ ID NO: 11, the HC-CDR2 comprising the amino acid sequence of SEQ ID NO: 12, and the HC-CDR3 comprising the amino acid sequence of SEQ ID NO: 13, and the V_{L-2} comprises the LC-CDR1 comprising the amino acid sequence of SEQ ID NO: 14, the LC-CDR2 comprising the amino acid sequence of SEQ ID NO: 15, and the LC-CDR3 comprising the amino acid sequence of SEQ ID NO: 16; or c) the V_{H-2} comprises the HC-CDR1 comprising the amino acid sequence of SEQ ID NO: 21, the HC-CDR2 comprising the amino acid sequence of SEQ ID NO: 22, and the HC-CDR3 comprising the amino acid sequence of SEQ ID NO: 23, and the V_{L-2} comprises the LC-CDR1 comprising the amino acid sequence of SEQ ID NO: 24, the LC-CDR2 comprising the amino acid sequence of SEQ ID NO: 25, and the LC-CDR3 comprising the amino acid sequence of SEQ ID NO: 26.

In some embodiments, the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 3, 2, or 1 amino acid substitutions in the HC-CDRs, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 3, 2, or 1 amino acid substitutions in the LC-CDRs.

In some embodiments, the V_{H} comprises i) the HC-CDR1 comprising the amino acid sequence of SEQ ID NO: 11, ii) the HC-CDR2 comprising the amino acid sequence of SEQ ID NO: 12, and iii) the HC-CDR3 comprising the amino acid sequence of SEQ ID NO: 13, or a variant thereof comprising up to 3, 2, or 1 amino acid substitutions in the HC-CDRs, and the V_{L} comprises i) the LC-CDR1 comprising the amino acid sequence of SEQ ID NO: 14, ii) the LC-CDR2 comprising the amino acid sequence of SEQ ID NO: 15, and iii) the LC-CDR3 comprising the amino acid sequence of SEQ ID NO: 16, or a variant thereof comprising up to 3, 2, or 1 amino acid substitutions in the LC-CDRs.

In some embodiments, the V_{H} comprises i) the HC-CDR1 comprising the amino acid sequence of SEQ ID NO: 21, ii) the HC-CDR2 comprising the amino acid sequence of SEQ ID NO: 22, and iii) the HC-CDR3 comprising the amino acid sequence of SEQ ID NO: 23, or a variant thereof comprising up to 3, 2, or 1 amino acid substitutions in the HC-CDRs, and the V_{L} comprises i) the LC-CDR1 comprising the amino acid sequence of SEQ ID NO: 24, ii) the LC-CDR2 comprising the amino acid sequence of SEQ ID NO: 25, and iii) the LC-CDR3 comprising the amino acid sequence of SEQ ID NO: 26, or a variant thereof comprising up to 3, 2, or 1 amino acid substitutions in the LC-CDRs.

In another aspect of the present application, there is provided an anti-B7-H4 construct comprising an antibody moiety that specifically binds to B7-H4, comprising: a) a heavy chain variable region (V_{H}) comprising a HC-CDR1, a HC-CDR2, and a HC-CDR3, respectively comprising the amino acid sequences of a CDR1, a CDR2, and a CDR3 within a V_{H} chain region having the sequence set forth in any one of SEQ ID NOs: 7, 17 and 27; and b) a light chain variable region (V_{L}) comprising a LC-CDR1, a LC-CDR2, and a LC-CDR3, respectively comprising the amino acid sequences of a CDR1, a CDR2, and a CDR3 within a V_{L}, chain region having the sequence set forth in any one of SEQ ID NOs: 8, 18, and 28.

In some embodiments according to any one of the anti-B7-H4 constructs described above, the V_{H} comprises an amino acid sequence of any one of SEQ ID NOs: 7, 17, and 27, or a variant comprising an amino acid sequence having at least about 80% sequence identity; and/or the V_{L} comprises an amino acid sequence of any one of SEQ ID NOs: 8, 18, and 28, or a variant comprising an amino acid sequence having at least about 80% sequence identity. In some embodiment, the V_{H} comprises an amino acid sequence of SEQ ID NO: 7, or a variant comprising an amino acid sequence having at least about 80% sequence identity; and the V_{L} comprises an amino acid sequence of SEQ ID NO: 8, or a variant comprising an amino acid sequence having at least about 80% sequence identity. In some embodiments, the V_{H} comprises an amino acid sequence of SEQ ID NO: 17, or a variant comprising an amino acid sequence having at least about 80% sequence identity; and wherein the V_{L} comprises an amino acid sequence of SEQ ID NO: 18, or a variant comprising an amino acid sequence having at least about 80% sequence identity. In some embodiments, the V_{H} comprises an amino acid sequence of SEQ ID NO: 27, or a variant comprising an amino acid sequence having at least about 80% sequence identity; and the V_{L} comprises an amino acid sequence of SEQ ID NO: 28, or a variant comprising an amino acid sequence having at least about 80% sequence identity.

In some embodiments according to any one of the anti-B7-H4 constructs described above, the construct is an antibody or antigen-binding fragment thereof selected from the group consisting of a full-length antibody, a bispecific antibody, a single-chain Fv (scFv) fragment, a Fab fragment, a Fab′ fragment, a F(ab′)2, an Fv fragment, a disulfide stabilized Fv fragment (dsFv), a (dsFv)2, a V_{H}H, a Fv-Fc fusion, a scFv-Fc fusion, a scFv-Fv fusion, a diabody, a tribody, and a tetrabody. In some embodiments, the construct comprises a full-length antibody.

In some embodiments according to any one of the anti-B7-H4 constructs described above, the antibody moiety has an Fc fragment of an immunoglobulin selected from the group consisting of IgG, IgA, lgD, IgE, IgM, and combinations and hybrids thereof. In some embodiments, the antibody moiety has an Fc fragment of an immunoglobulin selected from the group consisting of IgG1, IgG2, IgG3, IgG4, and combinations and hybrids thereof. In some embodiments, the Fc fragment has a reduced effector function as compared to the corresponding wildtype Fc fragment. In some embodiments, the Fc fragment has an enhanced effector function as compared to the corresponding wildtype Fc fragment.

In some embodiments according to any one of the anti-B7-H4 constructs described above, wherein the antibody moiety comprises a humanized antibody.

In some embodiments according to any one of the anti-B7-H4 constructs described above, the B7-H4 is a human B7-H4.

In another aspect of the present application, there is provided a pharmaceutical composition comprising any of the anti-B7-H4 constructs described above, and a pharmaceutical acceptable carrier.

In another aspect of the present application, there is provided an isolated nucleic acid encoding any of the anti-B7-H4 constructs described above.

In another aspect of the present application, there is provided a vector comprising any of the isolated nucleic acids described above.

In another aspect of the present application, there is provided an isolated host cell comprising any of the isolated nucleic acids or the vectors described above.

In another aspect of the present application, there is provided an immunoconjugate comprising any of the anti-B7-H4 construct described herein, linked to a therapeutic agent or a label.

In another aspect of the present application, there is provided a method of producing an anti-B7-H4 construct comprising: a) culturing any of the isolated host cells described above under conditions effective to express the anti-B7-H4 construct; and b) obtaining the expressed anti-B7-H4 construct from the host cell.

In another aspect of the present application, there is provided a method of treating a disease or condition in an individual, comprising administering to the individual an effective mount of any one of the anti-B7-H4 constructs or pharmaceutical compositions described above. In some embodiments, the disease or condition is a cancer. In some embodiments, the cancer is a solid tumor. In some embodiments, the cancer is a liquid tumor. In some embodiments, the cancer is a locally advanced or metastatic cancer. In some embodiments, the cancer is B7-H4-positive. In some embodiments, the cancer is selected from the group consisting of a lymphoma, colon cancer, breast cancer, ovarian cancer, endometrial cancer, esophageal cancer, prostate cancer, cervical cancer, renal cancer, bladder cancer, gastric cancer, non-small cell lung cancer, melanoma, and pancreatic cancer. In some embodiments, the anti-B7-H4 construct is administered parenterally into the individual. In some embodiments, the method further comprises administering a second agent. In some embodiments, the second agent comprises an immunomodulatory agent. In some embodiments, the immunomodulatory agent comprises an immune checkpoint inhibitor. In some embodiments, the immune checkpoint inhibitor comprises an anti-PD-1 antibody. In some embodiments, the immune checkpoint inhibitor is an anti-PD-L1 antibody. In some embodiments, the immunomodulatory agent comprises an immune co-stimulatory agonist. In some embodiments, the immune co-stimulatory agonist is an anti-4-1BB agonist. In some embodiments, the second agent is administered concurrently with the anti-B7-H4 agent. In some embodiments, the second agent is administered simultaneously with the anti-B7-H4 agent. In some embodiments, the second agent is administered sequentially with the anti-B7-H4 agent. In some embodiments, the individual is a human.

In another aspect of the present application, there is provided a method of modulating a cell composition, comprising contacting the cell composition with any one of the anti-B7-H4 constructs or pharmaceutical compositions. In some embodiments, the cell composition comprises T cells.

### BRIEF DESCRIPTION OF THE DRAWINGS

FIG. 1A shows binding of anti-B7-H4 antibodies (filled histogram) to human or mouse B7-H4 expressed on HEK-293T cells as analyzed by flow cytometry. Staining of antibodies to parental HEK-293T cells is depicted by the open histograms. M44, M50 and M100 share the same sequences.

FIG. 1B is a graph showing the binding of the indicated anti-B7-H4 antibodies at varying concentrations to human B7-H4 expressing HEK-293T cells, as analyzed by flow cytometry.

FIG. 1C is a graph showing the binding of the indicated anti-B7-H4 antibodies at varying concentrations to human B7-H4 naturally expressed on human SKBR3 cells, as analyzed by flow cytometry.

FIG. 1D is a graph showing the binding of the indicated anti-B7-H4 antibodies at varying concentrations to mouse B7-H4 expressing HEK-293T cells, as analyzed by flow cytometry.

FIG. 2 shows the levels of IL-2 or IFNγ produced by PBMCs stimulated with increasing amounts of staphylococcal enterotoxin B in the presence of fixed concentrations of anti-B7-H4 antibodies or control isotype antibody.

FIG. 3A shows the effect of anti-B7-H4 antibodies on CD4+ and CD8+ T cell proliferation.

FIG. 3B shows the effect of varying concentrations of anti-B7-H4 antibodies on CD4+ and CD8+ T cell proliferation.

FIG. 3C shows the effect of anti-B7-H4 antibodies on the production of IFNγ by T cells.

FIG. 4A shows the effect of anti-B7-H4 antibodies and an anti-PD-1 antibody as monotherapy on anti-tumor response of mice implanted with the E.G7-OVA tumor cell line (n=10/group). Treatment was commenced 5 days after tumor implantation.

FIG. 4B shows individual tumor volumes for E.G7-OVA bearing mice treated with the indicated anti-B7-H4 antibodies or anti-PD-1 antibody 24 days after tumor implantation.

FIG. 4C shows the individual tumor growth curves of mice implanted with E.G7-OVA tumor cells and treated with the indicated anti-B7-H4 antibodies or anti-PD-1 antibody as monotherapy. The number of tumor free animals is indicated for each treatment.

FIG. 5 shows the effect of concurrent administration of an anti-B7-H4 antibody (M44) and an anti-PD-1 antibody compared to the monotherapy treatment arms on the anti-tumor response of mice implanted with the MC38 tumor cell line (n=10/group). Treatment was commenced when tumors reached 20-85 mm³.

FIG. 6A shows the effect of concurrent administration of an anti-B7-H4 antibody (M44) and an anti-PD-1 antibody compared to the monotherapy treatment arms on the anti-tumor response of mice implanted with the A20 tumor cell line (n=9/group).

FIG. 6B shows the individual tumor growth curves of mice implanted with A20 tumor cells and treated with anti-B7-H4 antibody (M44) or anti-PD-1 antibody alone or in combination. The number of tumor free animals is indicated for each treatment.

FIG. 6C shows the survival of A20 tumor-bearing mice treated with anti-B7-H4 antibody (M44) or anti-PD-1 antibody alone or in combination.

### DETAILED DESCRIPTION OF THE APPLICATION

The present application provides novel anti-B7-H4 constructs that specifically bind to B7-H4 (such as anti-B7-H4 antibodies), methods of preparing the anti-B7-H4 constructs, methods of using the constructs (e.g., methods of treating a disease or condition, methods of modulating an immune response, or methods of modulating a cell composition).

### I. Definitions

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody moiety” refers to a full-length antibody or an antigen-binding fragment thereof.

A full-length antibody comprises two heavy chains and two light chains. The variable regions of the light and heavy chains are responsible for antigen binding. The variable domains of the heavy chain and light chain may be referred to as “V_{H}” and “V_{L}”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and p heavy chains, respectively. Several of the major antibody classes are divided into subclasses such as IgG1 (γ1 heavy chain), lgG2 (γ2 heavy chain), IgG3 (γ3 heavy chain), IgG4 (γ4 heavy chain), IgA1 (α1 heavy chain), or IgA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds.

In some embodiments, an antigen-binding fragment may comprise one or more CDRs from a particular human antibody grafted to a framework region from one or more different human antibodies.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacologv of Monoclonal Antibodies, vol. 113, Rosenburg and Moore eds., Springer-Verlag, New York, pp. 269-315 (1994).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol, 45: 3832-3839 (2008); Lefranc M. P. et al., Dev. Comp. Immunol., 27: 55-77 (2003); and Honegger and Plückthun, J Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or hypervariable region (HVR) of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

“Framework” or “FR” residues are those variable-domain residues other than the CDR residues as herein defined.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986): Riechmann et al., Nature 332:323-329 (1988); and Presta, Curr. Op. Struct. Biol. 2:593-596 (1992).

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol. 227:381 (1991); Marks et al., J Mol. Biol., 222:581 (1991). Also available for the preparation of human monoclonal antibodies are methods described in Cole et al., Monoclonal Antibodies and Cancer Therapy, Alan R Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or C_{L}) domain of the light chain.

The “light chains” of antibodies (immunoglobulins) from any mammalian species can be assigned to one of two clearly distinct types, called kappa (“κ”) and lambda (“λ”), based on the amino acid sequences of their constant domains.

The “CH1 domain” (also referred to as “C1” of “H1” domain) usually extends from about amino acid 118 to about amino acid 215 (EU numbering system).

“Hinge region” is generally defined as a region in IgG corresponding to Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region (also referred to as “C2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C2” domain) comprises the stretch of residues C-terminal to a CH2 domain in an Fc region (i.e. from about amino acid residue 341 to the C-terminal end of an antibody sequence, typically at amino acid residue 446 or 447 of an IgG).

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, and Fc-γRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med 126: 330-41 (1995). Other FcRs, including those to be identified in the future, are encompassed by the term “FcR” herein.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody or fragment thereof “competes” for binding to a target antigen with a second antibody or fragment thereof when the first antibody or fragment thereof inhibits the target antigen binding of the second antibody of fragment thereof by at least about 50% (such as at least about any one of 55%, 60%, 65%, 70%, 75%, 80%, 85%, 90%, 95%, 98% or 99%) in the presence of an equimolar concentration of the first antibody or fragment thereof, or vice versa. A high throughput process for “binning” antibodies based upon their cross-competition is described in PCT Publication No. WO 03/48731.

As use herein, the terms “specifically binds,” “specifically recognizing,” and “is specific for” refer to measurable and reproducible interactions, such as binding between a target and an antibody or antibody moiety, which is determinative of the presence of the target in the presence of a heterogeneous population of molecules, including biological molecules. For example, an antibody or antibody moiety that specifically recognizes a target (which can be an epitope) is an antibody or antibody moiety that binds this target with greater affinity, avidity, more readily, and/or with greater duration than its bindings to other targets. In some embodiments, the extent of binding of an antibody to an unrelated target is less than about 10% of the binding of the antibody to the target as measured, e.g., by a radioimmunoassay (RIA). In some embodiments, an antibody that specifically binds a target has a dissociation constant (K_{D}) of ≤10⁻⁵ M, ≥10⁻⁶ M, ≤10⁻⁷ M, ≤10⁻⁸ M, ≤10⁻⁹ M, ≥10⁻¹⁰ M, ≥10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA-, RIA-, ECL-, IRMA-, EIA-, BIACORE™-tests and peptide scans.

An “isolated” antibody (or construct) is one that has been identified, separated and/or recovered from a component of its production environment (e.g., natural or recombinant). Preferably, the isolated polypeptide is free of association with all other components from its production environment.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that have the same function or biological activity as screened or selected for in the originally transformed cell are included herein.

The term “immunoconjugate” includes reference to a covalent linkage of a therapeutic agent or a detectable label to an antibody such as an antibody moiety described herein. The linkage can be direct or indirect through a linker (such as a peptide linker).

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

In the context of cancer, the term “treating” includes any or all of: inhibiting growth of cancer cells, inhibiting replication of cancer cells, lessening of overall tumor burden and ameliorating one or more symptoms associated with the disease.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to that of a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual In some examples, a reference is obtained from one or more healthy individuals who are not the individual or patient.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing” as used herein, includes providing prophylaxis with respect to the occurrence or recurrence of a disease in an individual that may be predisposed to the disease but has not yet been diagnosed with the disease.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

The terms “subject,” “individual,” and “patient” are used interchangeably herein to refer to a mammal, including, but not limited to, human, bovine, horse, feline, canine, rodent, or primate. In some embodiments, the individual is a human.

An “effective amount” of an agent refers to an amount effective, at dosages and for periods of time necessary, to achieve the desired therapeutic or prophylactic result. The specific dose may vary depending on one or more of: the particular agent chosen, the dosing regimen to be followed, whether it is administered in combination with other compounds, timing of administration, the tissue to be imaged, and the physical delivery system in which it is carried.

A “therapeutically effective amount” of a substance/molecule of the application, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

The terms “pharmaceutical formulation” and “pharmaceutical composition” refer to a preparation which is in such form as to permit the biological activity of the active ingredient(s) to be effective, and which contains no additional components which are unacceptably toxic to an individual to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to an individual. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about 60 minutes, such as no more than about any of 30, 15, 10, 5, or 1 minutes.

The term “sequentially” is used herein to refer to administration of two or more therapeutic agents where the administration of one or more agent(s) continues after discontinuing the administration of one or more other agent(s). For example, administration of the two or more therapeutic agents are administered with a time separation of more than about 15 minutes, such as about any of 20, 30, 40, 50, or 60 minutes, 1 day, 2 days, 3 days, 1 week, 2 weeks, or 1 month, or longer.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain embodiments, the manufacture or kit is promoted, distributed, or sold as a unit for performing the methods described herein.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Anti-B7-H4 Constructs

The present application provides anti-B7-H4 constructs comprising an anti-B7-H4 antibody moiety that specifically binds to B7-H4 as described herein.

In some embodiments, the anti-B7-H4 construct comprises an antibody moiety comprising a heavy chain variable region (V_{H}) and a light chain variable region (Vt.), wherein the antibody moiety competes for a binding epitope of B7-H4 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6. In some embodiments, the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs. In some embodiments, the anti-B7-H4 antibody moiety is a humanized antibody derived from an anti-B7-H4 antibody comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6. In some embodiments, wherein the V_{H} comprises an amino acid sequence of SEQ ID NO: 7, or a variant comprising an amino acid sequence having at least about 80% (such as at least about any one of 80%, 85%, 90%, 95%, 96%, 97%, 98%, or 99%) sequence identity; and wherein the V_{L} comprises an amino acid sequence of SEQ ID NO: 8, or a variant comprising an amino acid sequence having at least about 80% (such as at least about any one of 80%, 85%, 90%, 95%, 96%, 97%, 98%, or 99%) sequence identity.

In some embodiments, the anti-B7-H4 construct comprises an antibody moiety comprising a heavy chain variable region (V_{H}) and a light chain variable region (V_{L}), wherein the antibody moiety competes for a binding epitope of B7-H4 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 11, the HC-CDR2 comprising the amino acid sequence of SEQ ID NO: 12, and the HC-CDR3 comprising the amino acid sequence of SEQ ID NO: 13, and the V_{L-2} comprises the LC-CDR1 comprising the amino acid sequence of SEQ ID NO: 14, the LC-CDR2 comprising the amino acid sequence of SEQ ID NO: 15, and the LC-CDR3 comprising the amino acid sequence of SEQ ID NO: 16. In some embodiments, the V_{H} comprises i) the HC-CDR1 comprising the amino acid sequence of SEQ ID NO: 11, ii) the HC-CDR2 comprising the amino acid sequence of SEQ ID NO: 12, and iii) the HC-CDR3 comprising the amino acid sequence of SEQ ID NO: 13, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 14, ii) the LC-CDR2 comprising the amino acid sequence of SEQ ID NO: 15, and iii) the LC-CDR3 comprising the amino acid sequence of SEQ ID NO: 16, or a variant thereof comprising up to 5, 4, 3, 2, or 1 amino acid substitutions in the LC-CDRs. In some embodiments, the anti-B7-H4 antibody moiety is a humanized antibody derived from an anti-B7-H4 antibody comprising a heavy chain variable region (V_{H}) and a light chain variable region (V_{L}), wherein the V_{H} comprises i) the HC-CDR1 comprising the amino acid sequence of SEQ ID NO: 11, ii) the HC-CDR2 comprising the amino acid sequence of SEQ ID NO: 12, and iii) the HC-CDR3 comprising the amino acid sequence of SEQ ID NO: 13, and the V_{L} comprises i) the LC-CDR1 comprising the amino acid sequence of SEQ ID NO: 14, ii) the LC-CDR2 comprising the amino acid sequence of SEQ ID NO: 15, and iii) the LC-CDR3 comprising the amino acid sequence of SEQ ID NO: 16. In some embodiments, wherein the V_{H} comprises an amino acid sequence of SEQ ID NO: 17, or a variant comprising an amino acid sequence having at least about 80% (such as at least about any one of 80%, 85%, 90%, 95%, 96%, 97%, 98%, or 99%) sequence identity; and wherein the V_{L} comprises an amino acid sequence of SEQ ID NO: 18, or a variant comprising an amino acid sequence having at least about 80% (such as at least about any one of 80%, 85%, 90%, 95%, 96%, 97%, 98%, or 99%) sequence identity.

In some embodiments, the anti-B7-H4 construct comprises an antibody moiety comprising a heavy chain variable region (V_{H}) and a light chain variable region (V_{L}), wherein the antibody moiety competes for a binding epitope of B7-H4 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 21, the HC-CDR2 comprising the amino acid sequence of SEQ ID NO: 22, and the HC-CDR3 comprising the amino acid sequence of SEQ ID NO: 23, and the V_{H-2} comprises the LC-CDR1 comprising the amino acid sequence of SEQ ID NO: 24, the LC-CDR2 comprising the amino acid sequence of SEQ ID NO: 25, and the LC-CDR3 comprising the amino acid sequence of SEQ ID NO: 26. In some embodiments, the V_{H} comprises i) the HC-CDR1 comprising the amino acid sequence of SEQ ID NO: 21, ii) the HC-CDR2 comprising the amino acid sequence of SEQ ID NO: 22, and iii) the HC-CDR3 comprising the amino acid sequence of SEQ ID NO: 23, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 24, ii) the LC-CDR2 comprising the amino acid sequence of SEQ ID NO: 25, and iii) the LC-CDR3 comprising the amino acid sequence of SEQ ID NO: 26, or a variant thereof comprising up to 5, 4, 3, 2, or 1 amino acid substitutions in the LC-CDRs. In some embodiments, the anti-B7-H4 antibody moiety is a humanized antibody derived from an anti-B7-H4 antibody comprising a heavy chain variable region (V_{H}) and a light chain variable region (V_{L}), wherein the V_{H} comprises i) the HC-CDR1 comprising the amino acid sequence of SEQ ID NO: 21, ii) the HC-CDR2 comprising the amino acid sequence of SEQ ID NO: 22, and iii) the HC-CDR3 comprising the amino acid sequence of SEQ ID NO: 23, and the V_{L} comprises i) the LC-CDR1 comprising the amino acid sequence of SEQ ID NO: 24, ii) the LC-CDR2 comprising the amino acid sequence of SEQ ID NO: 25, and iii) the LC-CDR3 comprising the amino acid sequence of SEQ ID NO: 26. In some embodiments, wherein the V_{H} comprises an amino acid sequence of SEQ ID NO: 27, or a variant comprising an amino acid sequence having at least about 80% (such as at least about any one of 80%, 85%, 90%, 95%, 96%, 97%, 98%, or 99%) sequence identity; and wherein the V_{L} comprises an amino acid sequence of SEQ ID NO: 28, or a variant comprising an amino acid sequence having at least about 80% (such as at least about any one of 80%, 85%, 90%, 95%, 96%, 97%, 98%, or 99%) sequence identity.

In some embodiments, there is provided an anti-B7-H4 construct comprising an antibody moiety that specifically binds to B7-H4, comprising: a) a heavy chain variable region (V_{H}) comprising a HC-CDR1, a HC-CDR2, and a HC-CDR3, respectively comprising the amino acid sequences of a CDR1, a CDR2, and a CDR3 within a V_{H} chain region having the sequence set forth in any one of SEQ ID NOs: 7, 17 and 27; and b) a light chain variable region (V_{L}) comprising a LC-CDR1, a LC-CDR2, and a LC-CDR3, respectively comprising the amino acid sequences of a CDR1, a CDR2, and a CDR3 within a V_{L} chain region having the sequence set forth in any one of SEQ ID NOs: 8, 18, and 28. In some embodiments, there is provided an anti-B7-H4 construct comprising an antibody moiety that specifically binds to B7-H4, comprising: a) a heavy chain variable region (V_{H}) comprising a HC-CDR1, a HC-CDR2, and a HC-CDR3, respectively comprising the amino acid sequences of a CDR1, a CDR2, and a CDR3 within a V_{H} chain region having the sequence set forth in any one of SEQ ID NOs: 7; and b) a light chain variable region (V_{L}) comprising a LC-CDR1, a LC-CDR2, and a LC-CDR3, respectively comprising the amino acid sequences of a CDR1, a CDR2, and a CDR3 within a V_{L} chain region having the sequence set forth in any one of SEQ ID NOs: 8. In some embodiments, wherein the V_{H} comprises an amino acid sequence of SEQ ID NO: 7, or a variant comprising an amino acid sequence having at least about 80% (such as at least about any one of 80%, 85%, 90%, 95%, 96%, 97%, 98%, or 99%) sequence identity; and wherein the V_{L} comprises an amino acid sequence of SEQ ID NO: 8, or a variant comprising an amino acid sequence having at least about 80% (such as at least about any one of 80%, 85%, 90%, 95%, 96%, 97%, 98%, or 99%) sequence identity.

In some embodiments, there is provided an anti-B7-H4 construct comprising an antibody moiety that specifically binds to B7-H4, comprising: a) a heavy chain variable region (V_{H}) comprising a HC-CDR1, a HC-CDR2, and a HC-CDR3, respectively comprising the amino acid sequences of a CDR1, a CDR2, and a CDR3 within a V_{H} chain region having the sequence set forth in any one of SEQ ID NOs: 17; and b) a light chain variable region (V_{L}) comprising a LC-CDR1, a LC-CDR2, and a LC-CDR3, respectively comprising the amino acid sequences of a CDR1, a CDR2, and a CDR3 within a V_{L} chain region having the sequence set forth in any one of SEQ ID NOs: 18. In some embodiments, wherein the V_{H} comprises an amino acid sequence of SEQ ID NO: 17, or a variant comprising an amino acid sequence having at least about 80% (such as at least about any one of 80%, 85%, 90%, 95%, 96%, 97%, 98%, or 99%) sequence identity; and wherein the V_{L} comprises an amino acid sequence of SEQ ID NO: 18, or a variant comprising an amino acid sequence having at least about 80% (such as at least about any one of 80%, 85%, 90%, 95%, 96%, 97%, 98%, or 99%) sequence identity.

In some embodiments, there is provided an anti-B7-H4 construct comprising an antibody moiety that specifically binds to B7-H4, comprising: a) a heavy chain variable region (V_{H}) comprising a HC-CDR1, a HC-CDR2, and a HC-CDR3, respectively comprising the amino acid sequences of a CDR1, a CDR2, and a CDR3 within a V_{H} chain region having the sequence set forth in any one of SEQ ID NOs: 27; and b) a light chain variable region (V_{L}) comprising a LC-CDR1, a LC-CDR2, and a LC-CDR3, respectively comprising the amino acid sequences of a CDR1, a CDR2, and a CDR3 within a V_{L} chain region having the sequence set forth in any one of SEQ ID NOs: 28. In some embodiments, wherein the V_{H} comprises an amino acid sequence of SEQ ID NO: 27, or a variant comprising an amino acid sequence having at least about 80% (such as at least about any one of 80%, 85%, 90%, 95%, 96%, 97%, 98%, or 99%) sequence identity; and wherein the V_{L} comprises an amino acid sequence of SEQ ID NO: 28, or a variant comprising an amino acid sequence having at least about 80% (such as at least about any one of 80%, 85%, 90%, 95%, 96%, 97%, 98%, or 99%) sequence identity.

In some embodiments, the construct comprises or is an antibody or antigen-binding fragment thereof selected from the group consisting of a full-length antibody, a bispecific antibody, a single-chain Fv (scFv) fragment, a Fab fragment, a Fab′ fragment, a F(ab′)2, an Fv fragment, a disulfide stabilized Fv fragment (dsFv), a (dsFv)2, a V_{H}H, a Fv-Fc fusion, a scFv-Fc fusion, a scFv-Fv fusion, a diabody, a tribody, and a tetrabody.

In some embodiments, the construct comprises or is a full-length antibody.

In some embodiments, the anti-B7-H4 antibody moiety or the full-length antibody described above comprises an Fc fragment of an immunoglobulin selected from the group consisting of IgG, IgA, IgD, IgE, IgM, and combinations and hybrids thereof. In some embodiments, the anti-B7-H4 antibody moiety or the full-length antibody described above comprises an Fc fragment of an immunoglobulin selected from the group consisting of IgG1, IgG2. IgG3, IgG4, and combinations and hybrids thereof. In some embodiments, the Fc fragment has a reduced effector function as compared to the corresponding wildtype Fc fragment. In some embodiments, the Fc fragment has an enhanced effector function as compared to the corresponding wildtype Fc fragment. In some embodiments, the antibody moiety or the full-length antibody described above comprises a humanized antibody.

In some embodiments, the anti-B7-H4 construct comprises or is an anti-B7-H4 fusion protein.

In some embodiments, the anti-B7-H4 construct comprises or is a multispecific anti-B7-H4 construct (such as a bispecific antibody).

In some embodiments, the anti-B7-H4 construct comprises or is an anti-B7-H4 immunoconjugate.

In some embodiments, the B7-H4 is a human B7-H4.

### Anti-B7-H4 Antibody Moieties

In some embodiments, the anti-B7-H4 antibody moiety comprises a heavy chain variable region (V_{H}) and a light chain variable region (V_{L}), wherein the antibody moiety competes for a binding epitope of B7-H4 with an antibody or antibody fragment comprising a second heavy variable region (V_{H-2}) and a second light chain variable region (V_{L-2}), wherein: a) the V_{H-2} comprises the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6; b) the V_{H-2} comprises the HC-CDR1 comprising the amino acid sequence of SEQ ID NO: 11, the HC-CDR2 comprising the amino acid sequence of SEQ ID NO: 12, and the HC-CDR3 comprising the amino acid sequence of SEQ ID NO: 13, and the V_{L-2} comprises the LC-CDR1 comprising the amino acid sequence of SEQ ID NO: 14, the LC-CDR2 comprising the amino acid sequence of SEQ ID NO: 15, and the LC-CDR3 comprising the amino acid sequence of SEQ ID NO: 16; or c) the V_{H-2} comprises the HC-CDR1 comprising the amino acid sequence of SEQ ID NO: 21, the HC-CDR2 comprising the amino acid sequence of SEQ ID NO: 22, and the HC-CDR3 comprising the amino acid sequence of SEQ ID NO: 23, and the V_{L-2} comprises the LC-CDR1 comprising the amino acid sequence of SEQ ID NO: 24, the LC-CDR2 comprising the amino acid sequence of SEQ ID NO: 25, and the LC-CDR3 comprising the amino acid sequence of SEQ ID NO: 26.

In some embodiments, the anti-B7-H4 antibody moiety comprises a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs.

In some embodiments, the anti-B7-H4 antibody moiety comprises a heavy chain variable region (V_{H}) and a light chain variable region (V_{L}), wherein the V_{H} comprises i) the HC-CDR1 comprising the amino acid sequence of SEQ ID NO: 11, ii) the HC-CDR2 comprising the amino acid sequence of SEQ ID NO: 12, and iii) the HC-CDR3 comprising the amino acid sequence of SEQ ID NO: 13, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 14, ii) the LC-CDR2 comprising the amino acid sequence of SEQ ID NO: 15, and iii) the LC-CDR3 comprising the amino acid sequence of SEQ ID NO: 16, or a variant thereof comprising up to 5, 4, 3, 2, or 1 amino acid substitutions in the LC-CDRs.

In some embodiments, the anti-B7-H4 antibody moiety comprises a heavy chain variable region (V_{H}) and a light chain variable region (V_{L}), wherein the V_{H} comprises i) the HC-CDR1 comprising the amino acid sequence of SEQ ID NO: 21, ii) the HC-CDR2 comprising the amino acid sequence of SEQ ID NO: 22, and iii) the HC-CDR3 comprising the amino acid sequence of SEQ ID NO: 23, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 24, ii) the LC-CDR2 comprising the amino acid sequence of SEQ ID NO: 25, and iii) the LC-CDR3 comprising the amino acid sequence of SEQ ID NO: 26, or a variant thereof comprising up to 5, 4, 3, 2, or 1 amino acid substitutions in the LC-CDRs.

In some embodiments, the amino acid substitutions are limited to “exemplary substitutions” shown in Table 2 of this application. In some embodiments, the amino acid substitutions are limited to “preferred substitutions” shown in Table 2 of this application.

In some embodiments, the anti-B7-H4 antibody moiety is a humanized antibody derived from an anti-B7-H4 antibody comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6.

In some embodiments, the anti-B7-H4 antibody moiety is a humanized antibody derived from an anti-B7-H4 antibody comprising a heavy chain variable region (V_{H}) and a light chain variable region (V_{L}), wherein the V_{H} comprises i) the HC-CDR1 comprising the amino acid sequence of SEQ ID NO: 11, ii) the HC-CDR2 comprising the amino acid sequence of SEQ ID NO: 12, and iii) the HC-CDR3 comprising the amino acid sequence of SEQ ID NO: 13, and the V_{L} comprises i) the LC-CDR1 comprising the amino acid sequence of SEQ ID NO: 14, ii) the LC-CDR2 comprising the amino acid sequence of SEQ ID NO: 15, and iii) the LC-CDR3 comprising the amino acid sequence of SEQ ID NO: 16.

In some embodiments, the anti-B7-H4 antibody moiety is a humanized antibody derived from an anti-B7-H4 antibody comprising a heavy chain variable region (V_{H}) and a light chain variable region (V_{L}), wherein the V_{H} comprises i) the HC-CDR1 comprising the amino acid sequence of SEQ ID NO: 21, ii) the HC-CDR2 comprising the amino acid sequence of SEQ ID NO: 22, and iii) the HC-CDR3 comprising the amino acid sequence of SEQ ID NO: 23, and the V_{L} comprises i) the LC-CDR1 comprising the amino acid sequence of SEQ ID NO: 24, ii) the LC-CDR2 comprising the amino acid sequence of SEQ ID NO: 25, and iii) the LC-CDR3 comprising the amino acid sequence of SEQ ID NO: 26.

In some embodiments, the anti-B7-H4 constructs described herein comprise an antibody moiety that specifically binds to B7-H4, comprising a) a heavy chain variable region (V_{H}) comprising a HC-CDR1, a HC-CDR2, and a HC-CDR3, respectively comprising the amino acid sequences of a CDR1, a CDR2, and a CDR3 within a V_{H} chain region having the sequence set forth in any one of SEQ ID NOs: 7, 17 and 27, and b) a light chain variable region (V_{L}) comprising a LC-CDR1, a LC-CDR2, and a LC-CDR3, respectively comprising the amino acid sequences of a CDR1, a CDR2, and a CDR3 within a V_{L} chain region having the sequence set forth in any one of SEQ ID NOs: 8, 18, and 28.

In some embodiments, the V_{H} comprises an amino acid sequence of any one of SEQ ID NOs: 7, 17, and 27, or a variant comprising an amino acid sequence having at least about 80% (such as at least about any one of 80%, 85%, 90%, 95%, 96%, 97%, 98%, or 99%) sequence identity; and/or wherein the V_{L} comprises an amino acid sequence of any one of SEQ ID NOs: 8, 18, and 28, or a variant comprising an amino acid sequence having at least about 80%% (such as at least about any one of 80%, 85%, 90%, 95%, 96%, 97%, 98%, or 99%) sequence identity.

a) Antibody Affinity

Binding specificity of the antibody moieties can be determined experimentally by methods known in the art. Such methods comprise, but are not limited to Western blots, ELISA-, RIA-, ECL-, IRMA-, EIA-, BIACORE™-tests and peptide scans.

In some embodiments, the K_{D} of the binding between the antibody moiety and B7-H4 is about 10⁻⁷ M to about 10⁻¹² M, about 10⁻⁷ M to about 10⁻⁸ M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁷ M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁷ M to about 10⁻¹¹ M, about 10⁻⁸ M to about 10⁻¹¹ M, about 10⁻⁹ M to about 10⁻¹¹ M, about 10⁻⁷ M to about 10⁻¹⁰ M, about 10⁻⁸ M to about 10⁻¹⁰ M, or about 10⁻⁷ M to about 10⁻⁹ M. In some embodiments, the K_{D} of the binding between the antibody moiety and B7-H4 is stronger than about any one of 10⁻⁷ M, 10⁻⁸ M, 10⁻⁹ M, 10⁻¹⁰ M, 10⁻¹¹ M, or 10⁻¹² M. In some embodiments, the B7-H4 is a human B7-H4.

In some embodiments, the Kₒₙ of the binding between the antibody moiety and B7-H4 is about 10³ M⁻¹s⁻¹ to about 10⁸ M⁻¹s⁻¹, about 10³ M⁻¹s⁻¹ to about 10⁴ M⁻¹s⁻¹, about 10⁴ M⁻¹s⁻¹ to about 10⁵ M⁻¹s⁻¹, about 10⁵M⁻¹s⁻¹ to about 10⁶ M⁻¹s⁻¹, about 10⁶ M⁻¹s⁻¹ to about 10⁷ M⁻¹s⁻¹, or about 10⁷ M⁻¹s⁻¹ to about 10⁸ M⁻¹s⁻¹. In some embodiments, the Kₒₙ of the binding between the antibody moiety and B7-H4 is about 10³ M⁻¹s⁻¹ to about 10⁵M⁻¹s⁻¹, about 10⁴ M⁻¹s⁻¹ to about 10⁶ M⁻¹s⁻¹, about 10⁵M⁻¹s⁻¹ to about 10⁷ M⁻¹s⁻¹, about 10⁶ M⁻¹s⁻¹ to about 10⁸ M⁻¹s⁻¹, about 10⁴ M⁻¹s⁻¹ to about 10⁷ M⁻¹s⁻¹, or about 10⁵M⁻¹s⁻¹ to about 10⁸ M⁻¹s⁻¹. In some embodiments, the Kₒₙ of the binding between the antibody moiety and B7-H4 is no more than about any one of 10³ M⁻¹s⁻¹, 10⁴ M⁻¹s⁻¹, 10⁵M⁻¹s⁻¹, 10⁶ M⁻¹s⁻¹, 10⁷ M⁻¹s⁻¹ or 10⁸ M⁻¹s⁻¹. In some embodiments, B7-H4 is human B7-H4.

In some embodiments, the K_{off} of the binding between the antibody moiety and B7-H4 is about 1 s⁻¹ to about 10⁻⁶ s⁻¹, about 1 s⁻¹ to about 10⁻² s⁻¹, about 10⁻² s⁻¹ to about 10⁻³ s⁻¹, about 10⁻³ s⁻¹ to about 10⁻⁴ s⁻¹, about 10⁻⁴ s⁻¹ to about 10⁻⁵ s⁻¹, about 10⁻⁵ s⁻¹ to about 10−⁶s⁻¹, about 1 s⁻¹ to about 10⁻⁵ s⁻¹, about 10⁻² s⁻¹ to about 10⁻⁶ s⁻¹, about 10⁻³ s⁻¹ to about 10⁻⁶ s⁻¹, about 10⁻⁴ s⁻¹ to about 10⁻⁶ s⁻¹, about 10⁻² s⁻¹ to about 10⁻⁵ s⁻¹, or about 10⁻³ s⁻¹ to about 10⁻⁵ s⁻¹. In some embodiments, the K_{off} of the binding between the antibody moiety and B7-H4 is at least about any one of 1 s⁻¹, 10⁻² s⁻¹, 10⁻³s⁻¹, 10⁻⁴ s⁻¹, 10⁻⁵ s⁻¹ or 10⁻⁶ s⁻¹. In some embodiments, B7-H4 is human B7-H4.

In some embodiments, the binding affinity of the anti-B7-H4 antibody moieties or anti-B7-H4 constructs are higher (for example, has a smaller K_{D} value) than an existing anti-B7-H4 antibody (e.g., anti-human B7-H4 antibody such as FPA150).

b) Chimeric or Humanized Antibodies

In some embodiments, the antibody moiety is a chimeric antibody. Certain chimeric antibodies are described, e.g., in U.S. Pat. No. 4,816,567; and Morrison et al., Proc. Natl. Acad. Sci. USA, 81:6851-6855 (1984)). In some embodiments, a chimeric antibody comprises a non-human variable region (e.g., a variable region derived from mouse) and a human constant region. In some embodiments, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see. e.g., Sims et al. J. Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

It is understood that the humanization of mouse derived antibodies is a common and routinely used art. It is therefore understood that a humanized format of any and all of the anti-B7-H4 antibodies disclosed in Sequence Table can be used in a preclinical or clinical setting. In cases where a humanized format of any of the referenced anti-B7-H4 antibodies or their antigen-binding regions thereof is used in such a preclinical or clinical setting, the then humanized format is expected to bear the same or similar biological activities and profiles as the original non-humanized format.

c) Human Antibodies

In some embodiments, the antibody moiety is a human antibody (known as human domain antibody, or human DAb). Human antibodies can be produced using various techniques known in the art. Human antibodies are described generally in van Dijk and van de Winkel, Curr. Opin. Pharmacol. 5: 368-74 (2001), Lonberg, Curr. Opin. Immunol. 20:450-459 (2008), and Chen, Mol. Immunol. 47(4):912-21 (2010). Transgenic mice or rats capable of producing fully human single-domain antibodies (or DAb) are known in the art. See, e.g., US20090307787A1, U.S. Pat. No. 8,754,287, US20150289489A1, US20100122358A1, and WO2004049794.

Human antibodies (e.g., human DAbs) may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 200710061900, describing VELOCIMOUSE® technology). Human variable regions from intact antibodies generated by such animals may be further modified, e.g., by combining with a different human constant region.

Human antibodies (e.g., human DAbs) can also be made by hybridoma-based methods. Human myeloma and mouse-human heteromyeloma cell lines for the production of human monoclonal antibodies have been described (See. e.g., Kozbor J. Immunol., 133: 3001 (1984); Brodeur et al., Monoclonal Antibody Production Techniques and Applications, pp. 51-63 (Marcel Dekker, Inc., New York, 1987), and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies (e.g., human DAbs) may also be generated by isolating Fv clone variable domain sequences selected from human-derived phage display libraries. Such variable domain sequences may then be combined with a desired human constant domain. Techniques for selecting human antibodies from antibody libraries are described below.

d) Library-Derived Antibodies

The antibody moieties may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004). Methods for constructing single-domain antibody libraries have been described, for example, see U.S. Pat. No. 7,371,849.

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J. 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

e) Substitution, Insertion, Deletion and Variants

In some embodiments, antibody variants having one or more amino acid substitutions are provided. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity. e.g., retained/improved antigen binding, decreased immunogenicity, or improved ADCC or CDC.

TABLE 2
Amino acid substitutions

| Original Residue | Exemplary Substitutions | Preferred Substitutions |
|---|---|---|
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y ) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln: (3) acidic: Asp, Glu: (4) basic: His, Lys, Arg; (5) residues that influence chain orientation: Gly, Pro; and (6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs. In some embodiments of the variant V_{H}H sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

f) Glycosylation Variants

In some embodiments, the antibody moiety is altered to increase or decrease the extent to which the construct is glycosylated. Addition or deletion of glycosylation sites to an antibody may be conveniently accomplished by altering the amino acid sequence such that one or more glycosylation sites is created or removed.

Where the antibody moiety comprises an Fc region (e.g., scFv-Fc), the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates. e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the antibody moiety may be made in order to create antibody variants with certain improved properties.

In some embodiments, the antibody moiety has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example, Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See. e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1. Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In some embodiments, the antibody moiety has bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.), and WO 1999/22764 (Raju, S.).

g) Fc Region Variants

In some embodiments, one or more amino acid modifications may be introduced into the Fc region of the antibody moiety (e.g., scFv-Fc), thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the Fc fragment possesses some but not all effector functions, which make it a desirable candidate for applications in which the half-life of the antibody moiety in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, 1, et al. Proc. Nat'l Acad Sci. USA 83:7059-7063 (1986)) and Hellstrom, I et al., Proc. Nat'l Acad Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). In some embodiments, the Fc fragment comprises a N297A mutation. In some embodiments, the Fc fragment comprises a N297G mutation.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, the Fc fragment is an IgG1 Fc fragment. In some embodiments, the IgG1 Fc fragment comprises a L234A mutation and/or a L235A mutation. In some embodiments, the Fc fragment is an IgG2 or IgG4 Fc fragment. In some embodiments, the Fc fragment is an IgG4 Fc fragment comprising a S228P, F234A, and/or a L235A mutation.

In some embodiments, the antibody moiety comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In some embodiments, the antibody moiety (e.g., scFv-Fc) variant comprising a variant Fc region comprising one or more amino acid substitutions which alters half-life and/or changes binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which alters binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

h) Cysteine Engineered Antibody Variants

In some embodiments, it may be desirable to create cysteine engineered antibody moieties, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: A118 (EU numbering) of the heavy chain; and S400 (EU numbering) of the heavy chain Fc region. Cysteine engineered antibody moieties may be generated as described, e.g., in U.S. Pat. No. 7,521,541.

i) Antibody Derivatives

In some embodiments, the antibody moiety described herein may be further modified to comprise additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in diagnosis under defined conditions, etc.

In some embodiments, the antibody moiety may be further modified to comprise one or more biologically active protein, polypeptides or fragments thereof. “Bioactive” or “biologically active”, as used herein interchangeably, means showing biological activity in the body to carry out a specific function. For example, it may mean the combination with a particular biomolecule such as protein, DNA, etc., and then promotion or inhibition of the activity of such biomolecule. In some embodiments, the bioactive protein or fragments thereof include proteins and polypeptides that are administered to patients as the active drug substance for prevention of or treatment of a disease or condition, as well as proteins and polypeptides that are used for diagnostic purposes, such as enzymes used in diagnostic tests or in vitro assays, as well as proteins and polypeptides that are administered to a patient to prevent a disease such as a vaccine.

### Anti-B7-H4 Fusion Proteins

The anti-B7-H4 constructs in some embodiments comprise an anti-B7-H4 antibody moiety and a second moiety, such as a half-life extending moiety. In some embodiments, the half-life extending moiety is an Fc fragment. In some embodiments, the half-life extending moiety is an albumin binding moiety (e.g., an albumin binding antibody moiety).

In some embodiments, the half-life extending moiety is an Fc fragment (such as any of the Fc fragments or variants thereof described herein). The term “Fc region,” “Fc domain” or “Fc” refers to a C-terminal non-antigen binding region of an immunoglobulin heavy chain that contains at least a portion of the constant region. The term includes native Fc regions and variant Fc regions. In some embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, Md., 1991.

In some embodiments, the Fc fragment is from an immunoglobulin selected from the group consisting of IgG, IgA, IgD, IgE, IgM, and combinations and hybrids thereof. In some embodiments, the Fc fragment is from an immunoglobulin selected from the group consisting of IgG1, IgG2, IgG3, IgG4, and combinations and hybrids thereof.

In some embodiments, the Fc fragment has a reduced effector function as compared to corresponding wildtype Fc fragment (such as at least about 30%, 40%, 50%, 60%, 70%, 80%, 85%, 90%, or 95% reduced effector function as measured by the level of antibody-dependent cellular cytotoxicity (ADCC)).

In some embodiments, the Fc fragment is an IgG1 Fc fragment. In some embodiments, the IgG1 Fc fragment comprises a L234A mutation and/or a L235A mutation. In some embodiments, the Fc fragment is an IgG2 or IgG4 Fc fragment. In some embodiments, the Fc fragment is an IgG4 Fc fragment comprising a S228P, F234A, and/or a L235A mutation. In some embodiments, the Fc fragment comprises a N297A mutation. In some embodiments, the Fc fragment comprises a N297G mutation.

In some embodiments, the anti-B7-H4 antibody moiety and the half-life extending moiety is linked via a linker (such as any of the linkers described in the “Linkers” section).

In some embodiments, the anti-B7-H4 fusion protein further comprises a second agent. In some embodiments, the second agent binds to a tumor agent (such as any one of the tumor agents described herein).

### Linkers

In some embodiments, the anti-B7-H4 constructs described herein comprise one or more linkers between two moieties (e.g., the anti-B7-H4 antibody moiety and the half-life extending moiety, the anti-B7-H4 antibody moiety and the second binding moiety in the multi-specific constructs described above). The length, the degree of flexibility and/or other properties of the linker(s) used in the bispecific antibodies may have some influence on properties, including but not limited to the affinity, specificity or avidity for one or more particular antigens or epitopes. For example, longer linkers may be selected to ensure that two adjacent domains do not sterically interfere with one another. In some embodiment, a linker (such as peptide linker) comprises flexible residues (such as glycine and serine) so that the adjacent domains are free to move relative to each other. For example, a glycine-serine doublet can be a suitable peptide linker. In some embodiments, the linker is a non-peptide linker. In some embodiments, the linker is a peptide linker. In some embodiments, the linker is a non-cleavable linker. In some embodiments, the linker is a cleavable linker.

Other linker considerations include the effect on physical or pharmacokinetic properties of the resulting compound, such as solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like.

### Peptide Linkers

The peptide linker may have a naturally occurring sequence, or a non-naturally occurring sequence. For example, a sequence derived from the hinge region of heavy chain only antibodies may be used as the linker. See, for example, WO1996/34103.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

An essential technical feature of such peptide linker is that said peptide linker does not comprise any polymerization activity. The characteristics of a peptide linker, which comprise the absence of the promotion of secondary structures, are known in the art and described, e.g., in Dall'Acqua et al. (Biochem. (1998) 37, 9266-9273), Cheadle et al. (Mol Immunol (1992) 29, 21-30) and Raag and Whitlow (FASEB (1995) 9(1), 73-80). A particularly preferred amino acid in context of the “peptide linker” is Gly. Furthermore, peptide linkers that also do not promote any secondary structures are preferred. The linkage of the domains to each other can be provided by, e.g., genetic engineering. Methods for preparing fused and operatively linked bispecific single chain constructs and expressing them in mammalian cells or bacteria are well-known in the art (e.g. WO 99/54440, Ausubel, Current Protocols in Molecular Biology, Green Publishing Associates and Wiley Interscience, N. Y. 1989 and 1994 or Sambrook et al., Molecular Cloning: A Laboratory Manual, Cold Spring Harbor Laboratory Press, Cold Spring Harbor, N. Y., 2001).

The peptide linker can be a stable linker, which is not cleavable by proteases, especially by Matrix metalloproteinases (MMPs).

The linker can also be a flexible linker. Exemplary flexible linkers include glycine polymers (G)ₙ(SEQ ID NO: 37), glycine-serine polymers (including, for example, (GS)ₙ (SEQ ID NO: 38), (GSGGS)ₙ (SEQ ID NO: 39), (GGGGS)ₙ (SEQ ID NO: 40), and (GGGS)ₙ (SEQ ID NO: 41), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). The ordinarily skilled artisan will recognize that design of an antibody fusion protein can include linkers that are all or partially flexible, such that the linker can include a flexible linker portion as well as one or more portions that confer less flexible structure to provide a desired antibody fusion protein structure.

Furthermore, exemplary linkers also include the amino acid sequence of such as (GGGGS)ₙ(SEQ ID NO: 40), wherein n is an integer between 1 and 8, e.g. (GGGGS)₃ (SEQ ID NO: 42, hereinafter referred to as “(G4S)3” or “GS3”), or (GGGGS)₆ (SEQ ID NO: 43; hereinafter referred to as “(G4S)6” or “GS6”). In some embodiments, the peptide linker comprises the amino acid sequence of (GSTSGSGKPGSGEGS)ₙ(SEQ ID NO: 44), wherein n is an integer between 1 and 3.

### Non-Peptide Linkers

Coupling of two moieties may be accomplished by any chemical reaction that will bind the two molecules so long as both components retain their respective activities, e.g., binding to B7-H4 and a second agent in a bispecific antibody, respectively. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents may be useful in coupling protein molecules in this context. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (see Killen and Lindstrom. Jour. Immun. 133:1335-2549 (1984); Jansen et al., Immunological Reviews 62:185-216 (1982); and Vitetta et al., Science 238:1098 (1987)).

Linkers the can be applied in the present application are described in the literature (see, for example, Ramakrishnan, S. et al., Cancer Res. 44:201-208 (1984) describing use of MBS (M-maleimidobenzoyl-N-hydroxysuccinimide ester). In some embodiments, non-peptide linkers used herein include: (i) EDC (1-ethyl-3-(3-dimethylamino-propyl) carbodiimide hydrochloride: (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido] hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus may lead to bispecific antibodies with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form antibody fusion protein with increased stability. Disulfide linkages, are in general, less stable than other linkages because the disulfide linkage is cleaved in vitro, resulting in less antibody fusion protein available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

### Multispecific Anti-B7-H4 Constructs

The anti-B7-H4 constructs in some embodiments comprise a multi-specific (e.g., bispecific) anti-B7-H4 construct comprising an anti-B7-H4 antibody moiety according to any one of the anti-B7-H4 antibody moieties described herein, and a second binding moiety (such as a second antibody moiety) specifically recognizing a second antigen. In some embodiments, the multi-specific anti-B7-H4 molecule comprises an anti-B7-H4 antibody moiety and a second antibody moiety specifically recognizing a second antigen. In some embodiments, the second antigen is a tumor associated antigen. In some embodiments, the second antigen is an immune checkpoint molecule. In some embodiments, the second antigen is PD-1 or PD-L1.

In some embodiments, the anti-B7-H4 construct is a multi-specific (e.g., bispecific) anti-B7-H4 construct comprising a) an anti-B7-H4 antibody moiety according to any one of the anti-B7-H4 antibody moieties described herein; b) a second binding moiety (such as a second antibody moiety) specifically recognizing a second antigen. In some embodiments, the second binding moiety and the anti-B7-H4 antibody moiety are fused with each other via a linker such as any of the linkers described herein with any operable form that allows the proper function of the binding moieties.

### Anti-B7-H4 Immunoconjugates

The present application also provides anti-B7-H4 immunoconjugates comprising an anti-B7-H4 antibody moiety (such as any of the B7-H4 antibody moiety) and a second agent. In some embodiments, the second agent is a therapeutic agent. In some embodiments, the second agent is a label.

In some embodiments, the second agent is a cytotoxic agent. In some embodiments, the cytotoxic agent is a chemotherapeutic agent. In some embodiments, the cytotoxic agent is a growth inhibitory agent. In some embodiments, the cytotoxic agent is a toxin (e.g., protein toxins, enzymatically active toxins of bacterial, fungal, plant, or animal origin, or fragments thereof). In some embodiments, the cytotoxic agent is a radioactive isotype (i.e., a radioconjugage).

Immunoconjugates allow for the targeted delivery of a drug moiety to a tumor, and, in some embodiments intracellular accumulation therein, where systemic administration of unconjugated drugs may result in unacceptable levels of toxicity to normal cells (Polakis P. (2005) Current Opinion in Pharmacology 5:382-387).

Antibody-drug conjugates (ADC) are targeted chemo therapeutic molecules which combine properties of both antibodies and cytotoxic drugs by targeting potent cytotoxic drugs to antigen-expressing tumor cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour: 14(3):154-169; Chari. R. V. (2008) ACC. Chen. Res. 41.98-107.

The ADC compounds of the invention include those with anticancer activity. In some embodiments, the ADC compounds include an antibody conjugated, i.e. covalently attached, to the drug moiety. In some embodiments, the antibody is covalently attached to the drug moiety through a linker. In some embodiments, the second agent is connected to the anti-B7-H4 antibody moiety via a linker (such as a linker described herein). In some embodiments, the linker is a cleavable. In some embodiments, the linker is non-cleavable.

The antibody-drug conjugates (ADC) of the application selectively deliver an effective dose of a drug to tumor tissue whereby greater selectivity. i.e. a lower efficacious dose, may be achieved while increasing the therapeutic index (“therapeutic window’). The drug moiety of the antibody-drug conjugates (ADC) may include any compound, moiety or group that has a cytotoxic or cytostatic effect. Drug moieties may impart their cytotoxic and cytostatic effects by mechanisms including but not limited to tubulin binding. DNA binding or intercalation, and inhibition of RNA polymerase, protein synthesis, and/or topoisomerase. Exemplary drug moieties include, but are not limited to, a mavtansinoid, dolastatin, auristatin, calicheamicin, pyrrolobenzodiazepine (PBD), nemorubicin and its derivatives, PNU-159682, anthracy cline, duocarmycin, Vinca alkaloid, taxane, trichothecene, CC1065, camptothecin, elinafide, and stereoisomers, isos teres, analogs, and derivatives thereof that have cytotoxic activity.

Production of immunoconjugates described herein can be found in, for example, U.S. Pat. Nos. 9,562,099 and 7,541,034, which are hereby incorporated by references in their entirety.

### Nucleic Acids

Nucleic acid molecules encoding the anti-B7-H4 constructs or anti-B7-H4 antibody moieties described herein are also contemplated. In some embodiments, there is provided a nucleic acid (or a set of nucleic acids) encoding a full-length anti-B7-H4 antibody. In some embodiments, there is provided a nucleic acid (or a set of nucleic acids) encoding an anti-B7-H4 antibody moiety. In some embodiments, there is provided a nucleic acid (or a set of nucleic acids) encoding an anti-B7-H4 Fc fusion protein. In some embodiments, there is provided a nucleic acid (or a set of nucleic acids) encoding a multi-specific anti-B7-H4 molecule (e.g., a multi-specific anti-B7-H4 construct), or polypeptide portion thereof. In some embodiments, the nucleic acid (or a set of nucleic acids) encoding the anti-B7-H4 construct described herein may further comprises a nucleic acid sequence encoding a peptide tag (such as protein purification tag, e.g., His-tag. HA tag).

Also contemplated here are isolated host cell comprising an anti-B7-H4 construct, an isolated nucleic acid encoding the polypeptide components of the anti-B7-H4 construct, or a vector comprising a nucleic acid encoding the polypeptide components of the anti-B7-H4 construct described herein.

The present application also includes variants to these nucleic acid sequences. For example, the variants include nucleotide sequences that hybridize to the nucleic acid sequences encoding the anti-B7-H4 constructs or anti-B7-H4 antibody moieties of the present application under at least moderately stringent hybridization conditions.

The present invention also provides vectors in which a nucleic acid of the present invention is inserted.

The nucleic acids of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/%584; WO 01/29058; and U.S. Pat. No. 6,326,193).

### III. Methods of Preparation

In some embodiments, there is provided a method of preparing an anti-B7-H4 construct or antibody moiety that specifically binds to B7-H4 and a composition such as polynucleotide, nucleic acid construct, vector, host cell, or culture medium that is produced during the preparation of the anti-B7-H4 construct or antibody moiety. The anti-B7-H4 construct or antibody moiety or composition described herein may be prepared by a number of processes as generally described below and more specifically in the Examples.

### Antibody Expression and Production

The antibodies (including anti-B7-H4 monoclonal antibodies, anti-B7-H4 bispecific antibodies, and anti-B7-H4 antibody moieties) described herein can be prepared using any known methods in the art, including those described below and in the Examples.

### Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies. For example, the monoclonal antibodies may be made using the hybridoma method first described by Kohler et al., Nature. 256:495 (1975), or may be made by recombinant DNA methods (U.S. Pat. No. 4,816,567). In the hybridoma method, a mouse or other appropriate host animal, such as a hamster or a llama, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986). Also see Example 1 for immunization in Camels.

The immunizing agent will typically include the antigenic protein or a fusion variant thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassas, Va. USA. Human myeloma and mouse-human heteromyeloma cell lines also have been described for the production of human monoclonal antibodies (Kozbor, J Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature. 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad Sci. USA, 81:6851 (1984)), or by covalently joining to the immunoglobulin coding sequence all or part of the coding sequence for a non-immunoglobulin poly peptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Nucleic Acid Molecules Encoding Antibody Moieties

In some embodiments, there is provided a polynucleotide encoding any one of the anti-B7-H4 constructs or antibody moieties described herein. In some embodiments, there is provided a polynucleotide prepared using any one of the methods as described herein. In some embodiments, a nucleic acid molecule comprises a polynucleotide that encodes a heavy chain or a light chain of an antibody moiety (e.g., anti-B7-H4 antibody moiety). In some embodiments, a nucleic acid molecule comprises both a polynucleotide that encodes a heavy chain and a polynucleotide that encodes a light chain, of an antibody moiety (e.g., anti-B7-H4 antibody moiety). In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody moiety (e.g., anti-B7-H4 antibody moiety) comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

In some embodiments, the polynucleotide is a DNA. In some embodiments, the polynucleotide is an RNA. In some embodiments, the RNA is an mRNA.

Nucleic acid molecules may be constructed using recombinant DNA techniques conventional in the art. In some embodiments, a nucleic acid molecule is an expression vector that is suitable for expression in a selected host cell.

### Nucleic Acid Construct

In some embodiments, there is provided a nucleic acid construct comprising any one of the polynucleotides described herein. In some embodiments, there is provided a nucleic acid construct prepared using any method described herein.

In some embodiments, the nucleic acid construct further comprises a promoter operably linked to the polynucleotide. In some embodiments, the polynucleotide corresponds to a gene, wherein the promoter is a wild-type promoter for the gene.

### Vectors

In some embodiments, there is provided a vector comprising any polynucleotides that encode the heavy chains and/or light chains of any one of the antibody moieties described herein (e.g., anti-B7-H4 antibody moieties) or nucleic acid construct described herein. In some embodiments, there is provided a vector prepared using any method described herein. Vectors comprising polynucleotides that encode any of anti-B7-H4 constructs such as antibodies, scFvs, fusion proteins or other forms of constructs described herein (e.g., anti-B7-H4 scFv) are also provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer el al., Biotechnol. Prog. 20:880-889 (2004).

### Host Cells

In some embodiments, there is provided a host cell comprising any polypeptide, nucleic acid construct and/or vector described herein. In some embodiments, there is provided a host cell prepared using any method described herein. In some embodiments, the host cell is capable of producing any of antibody moieties described herein under a fermentation condition.

In some embodiments, the antibody moieties described herein (e.g., anti-B7-H4 antibody moieties) may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PERC6® cells (Crucell); and NSO cells. In some embodiments, the antibody moieties described herein (e.g., anti-B7-H4 antibody moieties) may be expressed in yeast. See, e.g., U.S. Publication No. US 2006/0270045 A1. In some embodiments, a particular eukaryotic host cell is selected based on its ability to make desired post-translational modifications to the heavy chains and/or light chains of the antibody moiety. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection. DEAE-dextran mediated transfection, cationic lipid-mediated transfection, electroporation, transduction, infection, etc. Non-limiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

The invention also provides host cells comprising any of the polynucleotides or vectors described herein. In some embodiments, the invention provides a host cell comprising an anti-B7-H4 antibody. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S, pombe; or K. lactis).

In some embodiments, the antibody moiety is produced in a cell-free system. Non-limiting exemplary cell-free systems are described. e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### Culture Medium

In some embodiments, there is provided a culture medium comprising any antibody moiety, polynucleotide, nucleic acid construct, vector, and/or host cell described herein. In some embodiments, there is provided a culture medium prepared using any method described herein.

In some embodiments, the medium comprises hypoxanthine, aminopterin, and/or thymidine (e.g., HAT medium). In some embodiments, the medium does not comprise serum. In some embodiments, the medium comprises serum. In some embodiments, the medium is a D-MEM or RPMI-1640 medium.

### Purification of Antibody Moieties

The anti-B7-H4 constructs (e.g., anti-B7-H4 monoclonal antibodies or bispecific antibodies) may be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region and to purify an anti-B7-H4 construct comprising an Fc fragment. Hydrophobic interactive chromatography, for example, a butyl or phenyl column, may also suitable for purifying some polypeptides such as antibodies. Ion exchange chromatography (e.g. anion exchange chromatography and/or cation exchange chromatography) may also suitable for purifying some polypeptides such as antibodies. Mixed-mode chromatography (e.g. reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

### V. Methods of Treatments or Modulating an Immune Response in an Individual

Also provided here are methods of treating a disease or condition in an individual or modulating an immune response in an individual. The methods comprise administering the anti-B7-H4 construct described herein into individuals (e.g., mammals such as humans).

In some embodiments, there is provided a method of treating a disease or condition or modulating an immune response in an individual, comprising administering to the individual an effective amount of an anti-B7-H4 construct described herein.

In some embodiments, there is provided a method of treating a disease or condition in an individual, comprising administering to the individual an effective mount of the anti-B7-H4 construct comprising an antibody moiety comprising a heavy chain variable region (V_{H}) and a light chain variable region (V_{L}), wherein the antibody moiety competes for a binding epitope of B7-H4 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6. In some embodiments, the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 5, 4, 3, 2, or 1 amino acid substitutions in the LC-CDRs. In some embodiments, the anti-B7-H4 antibody moiety is a humanized antibody derived from an anti-B7-H4 antibody comprising a heavy chain variable region (V_{H}) and a light chain variable region (V_{L}), wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6. In some embodiments, wherein the V_{H} comprises an amino acid sequence of SEQ ID NO: 7, or a variant comprising an amino acid sequence having at least about 80% (such as at least about any one of 80%, 85%, 90%, 95%, 96%, 97%, 98%, or 99%) sequence identity; and wherein the V_{L} comprises an amino acid sequence of SEQ ID NO: 8, or a variant comprising an amino acid sequence having at least about 80% (such as at least about any one of 80%, 85%, 90%, 95%, 96%, 97%, 98%, or 99%) sequence identity.

In some embodiments, there is provided a method of treating a disease or condition in an individual, comprising administering to the individual an effective mount of the anti-B7-H4 construct comprising an antibody moiety comprising a heavy chain variable region (V_{H}) and a light chain variable region (V_{L}), wherein the antibody moiety competes for a binding epitope of B7-H4 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 11, the HC-CDR2 comprising the amino acid sequence of SEQ ID NO: 12, and the HC-CDR3 comprising the amino acid sequence of SEQ ID NO: 13, and the V_{L-2} comprises the LC-CDR1 comprising the amino acid sequence of SEQ ID NO: 14, the LC-CDR2 comprising the amino acid sequence of SEQ ID NO: 15, and the LC-CDR3 comprising the amino acid sequence of SEQ ID NO: 16. In some embodiments, the V_{H} comprises i) the HC-CDR1 comprising the amino acid sequence of SEQ ID NO: 11, ii) the HC-CDR2 comprising the amino acid sequence of SEQ ID NO: 12, and iii) the HC-CDR3 comprising the amino acid sequence of SEQ ID NO: 13, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 14, ii) the LC-CDR2 comprising the amino acid sequence of SEQ ID NO: 15, and iii) the LC-CDR3 comprising the amino acid sequence of SEQ ID NO: 16, or a variant thereof comprising up to 5, 4, 3, 2, or 1 amino acid substitutions in the LC-CDRs. In some embodiments, the anti-B7-H4 antibody moiety is a humanized antibody derived from an anti-B7-H4 antibody comprising a heavy chain variable region (V_{H}) and a light chain variable region (V_{L}), wherein the V_{H} comprises i) the HC-CDR1 comprising the amino acid sequence of SEQ ID NO: 11, ii) the HC-CDR2 comprising the amino acid sequence of SEQ ID NO: 12, and iii) the HC-CDR3 comprising the amino acid sequence of SEQ ID NO: 13, and the V_{L} comprises i) the LC-CDR1 comprising the amino acid sequence of SEQ ID NO: 14, ii) the LC-CDR2 comprising the amino acid sequence of SEQ ID NO: 15, and iii) the LC-CDR3 comprising the amino acid sequence of SEQ ID NO: 16. In some embodiments, wherein the V_{H} comprises an amino acid sequence of SEQ ID NO: 17, or a variant comprising an amino acid sequence having at least about 80% (such as at least about any one of 80%, 85%, 90%, 95%, 96%, 97%, 98%, or 99%) sequence identity; and wherein the V_{L} comprises an amino acid sequence of SEQ ID NO: 18, or a variant comprising an amino acid sequence having at least about 80% (such as at least about any one of 80%, 85%, 90%, 95%, 96%, 97%, 98%, or 99%) sequence identity.

In some embodiments, there is provided a method of treating a disease or condition in an individual, comprising administering to the individual an effective mount of the anti-B7-H4 construct comprising an antibody moiety comprising a heavy chain variable region (V_{H}) and a light chain variable region (V_{L}), wherein the antibody moiety competes for a binding epitope of B7-H4 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 21, the HC-CDR2 comprising the amino acid sequence of SEQ ID NO: 22, and the HC-CDR3 comprising the amino acid sequence of SEQ ID NO: 23, and the V_{L-2} comprises the LC-CDR1 comprising the amino acid sequence of SEQ ID NO: 24, the LC-CDR2 comprising the amino acid sequence of SEQ ID NO: 25, and the LC-CDR3 comprising the amino acid sequence of SEQ ID NO: 26. In some embodiments, the V_{H} comprises i) the HC-CDR1 comprising the amino acid sequence of SEQ ID NO: 21, ii) the HC-CDR2 comprising the amino acid sequence of SEQ ID NO: 22, and iii) the HC-CDR3 comprising the amino acid sequence of SEQ ID NO: 23, or a variant thereof comprising up to 5, 4, 3, 2, or 1 amino acid substitutions in the HC-CDRs, and the V_{L} comprises i) the LC-CDR1 comprising the amino acid sequence of SEQ ID NO: 24, ii) the LC-CDR2 comprising the amino acid sequence of SEQ ID NO: 25, and iii) the LC-CDR3 comprising the amino acid sequence of SEQ ID NO: 26, or a variant thereof comprising up to 5, 4, 3, 2, or 1 amino acid substitutions in the LC-CDRs. In some embodiments, the anti-B7-H4 antibody moiety is a humanized antibody derived from an anti-B7-H4 antibody comprising a heavy chain variable region (V_{H}) and a light chain variable region (V_{L}), wherein the V_{H} comprises i) the HC-CDR1 comprising the amino acid sequence of SEQ ID NO: 21, ii) the HC-CDR2 comprising the amino acid sequence of SEQ ID NO: 22, and iii) the HC-CDR3 comprising the amino acid sequence of SEQ ID NO: 23, and the V_{L} comprises i) the LC-CDR1 comprising the amino acid sequence of SEQ ID NO: 24, ii) the LC-CDR2 comprising the amino acid sequence of SEQ ID NO: 25, and iii) the LC-CDR3 comprising the amino acid sequence of SEQ ID NO: 26. In some embodiments, wherein the V_{H} comprises an amino acid sequence of SEQ ID NO: 27, or a variant comprising an amino acid sequence having at least about 80% (such as at least about any one of 80%, 85%, 90%, 95%, 96%, 97%, 98%, or 99%) sequence identity; and wherein the V_{L} comprises an amino acid sequence of SEQ ID NO: 28, or a variant comprising an amino acid sequence having at least about 80% (such as at least about any one of 80%, 85%, 90%, 95%, 96%, 97%, 98%, or 99%) sequence identity.

In some embodiments, the disease or condition is a cancer. In some embodiments, the cancer is a solid tumor. In some embodiments, the cancer is a liquid tumor. In some embodiments, the cancer is a locally advanced or metastatic cancer. In some embodiments, the cancer is B7-H4-positive. In some embodiments, the cancer is selected from the group consisting of a lymphoma, colon cancer, breast cancer, ovarian cancer, endometrial cancer, esophageal cancer, prostate cancer, cervical cancer, renal cancer, bladder cancer, gastric cancer, non-small cell lung cancer, melanoma, and pancreatic cancer. In some embodiments, wherein the anti-B7-H4 construct is administered parenterally (e.g., intravenously) into the individual.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or genetically prone to one or more of the diseases or disorders described herein (such as a cancer, an autoimmune disease). In some embodiments, the individual has one or more risk factors associated with one or more diseases or disorders described herein.

### Modulating Immune Response

In some embodiments, the modulating of immune response comprises modulating a cell population in the individual. In some embodiments, the cell population comprises a T cell population. In some embodiments, the T cell population comprises a CD4+ T cell population. In some embodiments, the T cell population comprises a CD8+ T cell population. In some embodiments, the cell population comprises myeloid derived suppressor cells. In some embodiments, the cell population comprises neutrophils.

In some embodiments, there is provided a method of modulating a cell composition, comprising contacting the cell composition (e.g., a T cell population, e.g., a CD4+ and/or CD8+ T cell population) with any of the anti-B7-H4 constructs or pharmaceutical compositions described herein.

In some embodiments, the modulating comprises promoting proliferation of a cell population (e.g., a T cell population, e.g., a CD4+ and/or CD8+ T cell population). In some embodiments, the proliferation of the cell population after the contact with the anti-B7-H4 construct is increased by at least about 20%, 30%, 40%, 50%, 60%, 70%, 80%, or 90% as compared to the proliferation of the reference cells after the contact with a control construct. In some embodiments, the proliferation of the cell population after the contact with the anti-B7-H4 construct is increased by at least about 1-fold, 1.2-fold, 1.5 fold, 1.7-fold, 2-fold, 2.5-fold, 3-fold, 3.5-fold, 4-fold, 4.5-fold, 5-fold, 5.5-fold, or 6-fold as compared to the proliferation of the reference cells after the contact with a control construct. In some embodiments, the control construct comprises or is a reference antibody that also binds to B7-H4 (such as FPA150). In some embodiments, the control construct comprises or is an antibody that does not bind to anti-B7-H4.

In some embodiments, the modulating comprises promoting cytokine production of a cell population (e.g., a T cell population, e.g., a CD4+ and/or CD8+ T cell population). In some embodiments, the cytokine production of the cell population after the contact with the anti-B7-H4 construct is increased by at least about 20%, 30%, 40%, 50%, 60%, 70%, 80%, or 90% as compared to the proliferation of the reference cells after the contact with a control construct. In some embodiments, the proliferation of the cell population after the contact with the anti-B7-H4 construct is increased by at least about 1-fold, 1.2-fold, 1.5 fold, 1.7-fold. 2-fold, 2.5-fold, 3-fold, 3.5-fold, 4-fold, 4.5-fold, 5-fold, 5.5-fold, or 6-fold as compared to the proliferation of the reference cells after the contact with a control construct. In some embodiments, the control construct comprises or is a reference antibody that also binds to B7-H4 (such as FPA150). In some embodiments, the control construct comprises or is an antibody that does not bind to anti-B7-H4.

### Dosing and Method of Administering the Anti-B7-H4 Construct

The dosing regimen of the anti-B7-H4 construct (such as the specific dosages and frequencies) used for treating a disease or disorder as described herein administered into the individual may vary with the particular anti-B7-H4 construct (such as anti-B7-H4 monoclonal or multispecific antibodies, such as anti-B7-H4 fusion proteins), the mode of administration, and the type of disease or condition being treated. In some embodiments, the type of disease or condition is a cancer. In some embodiments, the effective amount of the anti-B7-H4 construct (such as anti-B7-H4 monoclonal or multispecific antibodies) is an amount that is effective to result in an objective response (such as a partial response or a complete response). In some embodiments, the effective amount of the anti-B7-H4 construct (such as anti-B7-H4 monoclonal or multispecific antibodies) is an amount that is sufficient to result in a complete response in the individual. In some embodiments, the effective amount of the anti-B7-H4 construct (such as anti-B7-H4 monoclonal or multispecific antibodies) is an amount that is sufficient to result in a partial response in the individual. In some embodiments, the effective amount of anti-B7-H4 construct (such as anti-B7-H4 monoclonal or multispecific antibodies) is an amount that is sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the anti-B7-H4 construct (such as anti-B7-H4 monoclonal or multispecific antibodies). Responses of an individual to the treatment of the methods described herein can be determined, for example, based on RECIST levels.

In some embodiments, the effective amount of the anti-B7-H4 construct (such as anti-B7-H4 monoclonal or multispecific antibodies) is an amount that is sufficient to prolong progress-free survival of the individual. In some embodiments, the effective amount of the anti-B7-H4 construct (such as anti-B7-H4 monoclonal or multispecific antibodies) is an amount that is sufficient to prolong overall survival of the individual. In some embodiments, the effective amount of the anti-B7-H4 construct (such as anti-B7-H4 monoclonal or multispecific antibodies) is an amount that is sufficient to produce clinical benefit of more than about any of 50%, 60%, 70%, or 77% among a population of individuals treated with the anti-B7-H4 construct (such as anti-B7-H4 monoclonal or multispecific antibodies).

In some embodiments, the effective amount of the anti-B7-H4 construct (such as anti-B7-H4 monoclonal or multispecific antibodies) alone or in combination with a second, third, and/or fourth agent, is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment (e.g., receiving a placebo treatment). Standard methods can be used to measure the magnitude of this effect, such as in vitro assays with purified enzyme, cell-based assays, animal models, or human testing.

In some embodiments, the effective amount of the anti-B7-H4 construct (such as anti-B7-H4 monoclonal or multispecific antibodies) is an amount that is below the level that induces a toxicological effect (i.e., an effect above a clinically acceptable level of toxicity) or is at a level where a potential side effect can be controlled or tolerated when the composition is administered to the individual.

In some embodiments, the effective amount of the anti-B7-H4 construct (such as anti-B7-H4 monoclonal or multispecific antibodies) is an amount that is close to a maximum tolerated dose (MTD) of the composition following the same dosing regimen. In some embodiments, the effective amount of the anti-B7-H4 construct (such as anti-B7-H4 monoclonal or multispecific antibodies) is more than about any of 80%, 90%, 95%, or 98% of the MTD.

In some embodiments, the effective amount of the anti-B7-H4 construct (such as anti-B7-H4 monoclonal or multispecific antibodies) is an amount that slows or inhibits the progression of the disease or condition (for example, by at least about 5%, 10%, 15%, 20%, 30%, 40%, 50%) as compared to that of the individual not receiving the treatment. In some embodiments, the disease or condition is an autoimmune disease. In some embodiments, the disease or condition is an infection.

In some embodiments, the effective amount of the anti-B7-H4 construct (such as anti-B7-H4 monoclonal or multispecific antibodies) is an amount that reduces the side effects (autoimmune response) of a condition (e.g., transplantation)(for example, by at least about 5%, 10%, 15%, 20%, 30%, 40%, or 50%) as compared to that of the individual not receiving the treatment.

In some embodiments of any of the above aspects, the effective amount of an anti-B7-H4 construct (such as anti-B7-H4 monoclonal or multispecific antibodies) is in the range of about 0.001 μg/kg to about 100 mg/kg of total body weight, for example, about 0.005 μg/kg to about 50 mg/kg, about 0.01 μg/kg to about 10 mg/kg, or about 0.01 μg/kg to about 1 mg/kg.

In some embodiments, the treatment comprises more than one administration of the anti-B7-H4 constructs (such as about two, three, four, five, six, seven, eight, night, or ten administrations of anti-B7-H4 constructs). In some embodiments, two administrations are carried out within about a week. In some embodiments, a second administration is carried out at least about 1, 2, 3, 4, 5, 6, or 7 days after the completion of the first administration. In some embodiments, a second administration is carried out about 1-14 days, 1-10 days, 1-7 days, 2-6 days, or 3-5 days after the completion of the first administration. In some embodiments, the anti-B7-H4 construct is administered about 1-3 times a week (such as about once a week, about twice a week, or about three times a week).

The anti-B7-H4 construct can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, the anti-B7-H4 construct is included in a pharmaceutical composition while administered into the individual. In some embodiments, sustained continuous release formulation of the composition may be used. In some embodiments, the composition is administered intravenously. In some embodiments, the composition is administered intraperitoneally. In some embodiments, the composition is administered intravenously. In some embodiments, the composition is administered intraperitoneally. In some embodiments, the composition is administered intramuscularly. In some embodiments, the composition is administered subcutaneously. In some embodiments, the composition is administered intravenously. In some embodiments, the composition is administered orally.

### Combination Therapy

This application also provides methods of administering an anti-B7-H4 construct into an individual for treating a disease or condition (such as cancer), wherein the method further comprises administering a second agent or therapy. In some embodiments, the second agent or therapy is a standard or commonly used agent or therapy for treating the disease or condition. In some embodiments, the second agent or therapy comprises a chemotherapeutic agent. In some embodiments, the second agent or therapy comprises a surgery. In some embodiments, the second agent or therapy comprises a radiation therapy. In some embodiments, the second agent or therapy comprises an immunotherapy. In some embodiments, the second agent or therapy comprises a hormonal therapy. In some embodiments, the second agent or therapy comprises an angiogenesis inhibitor. In some embodiments, the second agent or therapy comprises a tyrosine kinase inhibitor. In some embodiments, the second agent or therapy comprises an infectious agent.

In some embodiments, the second agent or therapy comprises an immunomodulatory agent. In some embodiments, the immunomodulatory agent comprises an immune checkpoint inhibitor. In some embodiments, the immune checkpoint inhibitor comprises an anti-PD-1 antibody. In some embodiments, the immune checkpoint inhibitor comprises an anti-PD-1 antibody.

In some embodiments, the second agent or therapy targets an immune costimulatory molecule (such as 4-1 BB).

In some embodiments, the anti-B7-H4 construct is administered simultaneously with the second agent or therapy. In some embodiments, the anti-B7-H4 construct is administered concurrently with the second agent or therapy. In some embodiments, the anti-B7-H4 construct is administered sequentially with the second agent or therapy. In some embodiments, the anti-B7-H4 construct is administered prior to the second agent or therapy. In some embodiments, the anti-B7-H4 construct is administered after the second agent or therapy. In some embodiments, the anti-B7-H4 construct is administered in the same unit dosage form as the second agent or therapy. In some embodiment, the anti-B7-H4 construct is administered in a different unit dosage form from the second agent or therapy.

### VI. Compositions, Kits and Articles of Manufacture

Also provided herein are compositions (such as formulations) comprising any one of the anti-B7-H4 construct or anti-B7-H4 antibody moiety described herein, nucleic acid encoding the antibody moieties, vector comprising the nucleic acid encoding the antibody moieties, or host cells comprising the nucleic acid or vector.

Suitable formulations of the anti-B7-H4 construct described herein can be obtained by mixing the anti-B7-H4 construct or anti-B7-H4 antibody moiety having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzvl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine: monosaccharides, disaccharides, and other carbohydrates including glucose, mannose, or dextrins: chelating agents such as EDTA: sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be imaged, diagnosed, or treated herein.

The formulations to be used for in vivo administration must be sterile. This is readily accomplished by, e.g., filtration through sterile filtration membranes.

Also provided are kits comprising any one of the anti-B7-H4 construct or anti-B7-H4 antibody moiety described herein. The kits may be useful for any of the methods of modulating cell composition or treatment described herein.

In some embodiments, there is provided a kit comprising an anti-B7-H4 construct specifically binding to B7-H4.

In some embodiments, the kit further comprises a device capable of delivering the anti-B7-H4 construct into an individual. One type of device, for applications such as parenteral delivery, is a syringe that is used to inject the composition into the body of a subject. Inhalation devices may also be used for certain applications.

In some embodiments, the kit further comprises a therapeutic agent for treating a disease or condition, e.g., cancer, infectious disease, autoimmune disease, or transplantation.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information.

The present application thus also provides articles of manufacture. The article of manufacture can comprise a container and a label or package insert on or associated with the container. Suitable containers include vials (such as sealed vials), bottles, jars, flexible packaging, and the like. Generally, the container holds a composition, and may have a sterile access port (for example the container may be an intravenous solution bag or a vial having a stopper pierceable by a hypodermic injection needle). The label or package insert indicates that the composition is used for imaging, diagnosing, or treating a particular condition in an individual. The label or package insert will further comprise instructions for administering the composition to the individual and for imaging the individual. The label may indicate directions for reconstitution and/or use. The container holding the composition may be a multi-use vial, which allows for repeat administrations (e.g. from 2-6 administrations) of the reconstituted formulation. Package insert refers to instructions customarily included in commercial packages of diagnostic products that contain information about the indications, usage, dosage, administration, contraindications and/or warnings concerning the use of such diagnostic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the compositions and instructions for use, packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXEMPLARY EMBODIMENTS

Embodiment 1. An anti-B7-H4 construct comprising an antibody moiety comprising a heavy chain variable region (V_{H}) and a light chain variable region (V_{L}), wherein the antibody moiety competes for a binding epitope of B7-H4 with an antibody or antibody fragment comprising a second heavy variable region (V_{H-2}) and a second light chain variable region (V_{L-2}), wherein: a) the V_{H-2} comprises the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6; b) the V_{H-2} comprises the HC-CDR1 comprising the amino acid sequence of SEQ ID NO: 11, the HC-CDR2 comprising the amino acid sequence of SEQ ID NO: 12, and the HC-CDR3 comprising the amino acid sequence of SEQ ID NO: 13, and the V_{L-2} comprises the LC-CDR1 comprising the amino acid sequence of SEQ ID NO: 14, the LC-CDR2 comprising the amino acid sequence of SEQ ID NO: 15, and the LC-CDR3 comprising the amino acid sequence of SEQ ID NO: 16; or c) the V_{H-2} comprises the HC-CDR1 comprising the amino acid sequence of SEQ ID NO: 21, the HC-CDR2 comprising the amino acid sequence of SEQ ID NO: 22, and the HC-CDR3 comprising the amino acid sequence of SEQ ID NO: 23, and the VI-2 comprises the LC-CDR1 comprising the amino acid sequence of SEQ ID NO: 24, the LC-CDR2 comprising the amino acid sequence of SEQ ID NO: 25, and the LC-CDR3 comprising the amino acid sequence of SEQ ID NO: 26.

Embodiment 2. The anti-B7-H4 construct of embodiment 1, wherein: a) the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 3, 2, or 1 amino acid substitutions in the HC-CDRs, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 3, 2, or 1 amino acid substitutions in the LC-CDRs; b) the V_{H} comprises i) the HC-CDR1 comprising the amino acid sequence of SEQ ID NO: 11, ii) the HC-CDR2 comprising the amino acid sequence of SEQ ID NO: 12, and iii) the HC-CDR3 comprising the amino acid sequence of SEQ ID NO: 13, or a variant thereof comprising up to 3, 2, or 1 amino acid substitutions in the HC-CDRs, and the V_{L} comprises i) the LC-CDR1 comprising the amino acid sequence of SEQ ID NO: 14, ii) the LC-CDR2 comprising the amino acid sequence of SEQ ID NO: 15, and iii) the LC-CDR3 comprising the amino acid sequence of SEQ ID NO: 16, or a variant thereof comprising up to 3, 2, or 1 amino acid substitutions in the LC-CDRs; or c) the V_{H} compnses i) the HC-CDR1 comprising the amino acid sequence of SEQ ID NO: 21, ii) the HC-CDR2 comprising the amino acid sequence of SEQ ID NO: 22, and iii) the HC-CDR3 comprising the amino acid sequence of SEQ ID NO: 23, or a variant thereof comprising up to 3, 2, or 1 amino acid substitutions in the HC-CDRs, and the V_{L} comprises i) the LC-CDR1 comprising the amino acid sequence of SEQ ID NO: 24, ii) the LC-CDR2 comprising the amino acid sequence of SEQ ID NO: 25, and iii) the LC-CDR3 comprising the amino acid sequence of SEQ ID NO: 26, or a variant thereof comprising up to 3, 2, or 1 amino acid substitutions in the LC-CDRs.

Embodiment 3. The anti-B7-H4 construct of embodiment 2, wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 3, 2, or 1 amino acid substitutions in the HC-CDRs; and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 3, 2, or 1 amino acid substitutions in the LC-CDRs.

Embodiment 4. The anti-B7-H4 construct of embodiment 2, wherein the V_{H} comprises i) the HC-CDR1 comprising the amino acid sequence of SEQ ID NO: 11, ii) the HC-CDR2 comprising the amino acid sequence of SEQ ID NO: 12, and iii) the HC-CDR3 comprising the amino acid sequence of SEQ ID NO: 13, or a variant thereof comprising up to 3, 2, or 1 amino acid substitutions in the HC-CDRs; and the V_{L} comprises i) the LC-CDR1 comprising the amino acid sequence of SEQ ID NO: 14, ii) the LC-CDR2 comprising the amino acid sequence of SEQ ID NO: 15, and iii) the LC-CDR3 comprising the amino acid sequence of SEQ ID NO: 16, or a variant thereof comprising up to 3, 2, or 1 amino acid substitutions in the LC-CDRs.

Embodiment 5. The anti-B7-H4 construct of embodiment 2, wherein the V_{H} comprises i) the HC-CDR1 comprising the amino acid sequence of SEQ ID NO: 21, ii) the HC-CDR2 comprising the amino acid sequence of SEQ ID NO: 22, and iii) the HC-CDR3 comprising the amino acid sequence of SEQ ID NO: 23, or a variant thereof comprising up to 3, 2, or 1 amino acid substitutions in the HC-CDRs; and the V_{L} comprises i) the LC-CDR1 comprising the amino acid sequence of SEQ ID NO: 24, ii) the LC-CDR2 comprising the amino acid sequence of SEQ ID NO: 25, and iii) the LC-CDR3 comprising the amino acid sequence of SEQ ID NO: 26, or a variant thereof comprising up to 3, 2, or 1 amino acid substitutions in the LC-CDRs.

Embodiment 6. An anti-B7-H4 construct comprising an antibody moiety that specifically binds to B7-H4, comprising: a) a heavy chain variable region (V_{H}) comprising a HC-CDR1, a HC-CDR2, and a HC-CDR3, respectively comprising the amino acid sequences of a CDR1, a CDR2, and a CDR3 within a V_{H} chain region having the sequence set forth in any one of SEQ ID NOs: 7, 17 and 27; and b) a light chain variable region (V_{L}) comprising a LC-CDR1, a LC-CDR2, and a LC-CDR3, respectively comprising the amino acid sequences of a CDR1, a CDR2, and a CDR3 within a V_{L} chain region having the sequence set forth in any one of SEQ ID NOs: 8, 18, and 28.

Embodiment 7. The anti-B7-H4 construct of any one of embodiments 1-6, wherein the V_{H} comprises an amino acid sequence of any one of SEQ ID NOs: 7, 17, and 27, or a variant comprising an amino acid sequence having at least about 80% sequence identity; and/or wherein the V_{L} comprises an amino acid sequence of any one of SEQ ID NOs: 8, 18, and 28, or a variant comprising an amino acid sequence having at least about 80% sequence identity.

Embodiment 8. The anti-B7-H4 construct of embodiment 7, wherein: 1) the V_{H} comprises an amino acid sequence of SEQ ID NO: 7, or a variant comprising an amino acid sequence having at least about 80% sequence identity, and wherein the V_{L} comprises an amino acid sequence of SEQ ID NO: 8, or a variant comprising an amino acid sequence having at least about 80% sequence identity; 2) the V_{H} comprises an amino acid sequence of SEQ ID NO: 17, or a variant comprising an amino acid sequence having at least about 80% sequence identity; and wherein the V_{L} comprises an amino acid sequence of SEQ ID NO: 18, or a variant comprising an amino acid sequence having at least about 80% sequence identity; or 3) the V_{H} comprises an amino acid sequence of SEQ ID NO: 27, or a variant comprising an amino acid sequence having at least about 80% sequence identity; and wherein the V_{L} comprises an amino acid sequence of SEQ ID NO: 28, or a variant comprising an amino acid sequence having at least about 80% sequence identity.

Embodiment 9. The anti-B7-H4 construct of any one of embodiments 1-8, wherein the construct is an antibody or antigen-binding fragment thereof selected from the group consisting of a full-length antibody, a bispecific antibody, a single-chain Fv (scFv) fragment, a Fab fragment, a Fab′ fragment, a F(ab′)2, an Fv fragment, a disulfide stabilized Fv fragment (dsFv), a (dsFv)2, a V_{H}H, a Fv-Fc fusion, a scFv-Fc fusion, a scFv-Fv fusion, a diabody, a tribody, and a tetrabody.

Embodiment 10. The anti-B7-H4 construct of embodiment 9, wherein the construct comprises a full-length antibody.

Embodiment 11. The anti-B7-H4 construct of any one of embodiments 1-10, wherein the antibody moiety has an Fc fragment of an immunoglobulin selected from the group consisting of IgG, IgA, IgD, IgE, IgM, and combinations and hybrids thereof.

Embodiment 12. The anti-B7-H4 construct of embodiment 11, wherein the antibody moiety has an Fc fragment of an immunoglobulin selected from the group consisting of IgG1, IgG2, IgG3, IgG4, and combinations and hybrids thereof.

Embodiment 13. The anti-B7-H4 construct of embodiment 11 or embodiment 12, wherein the Fc fragment has a reduced effector function as compared to the corresponding wildtype Fc fragment.

Embodiment 14. The anti-B7-H4 construct of embodiment 11 or embodiment 12, wherein the Fc fragment has an enhanced effector function as compared to the corresponding wildtype Fc fragment.

Embodiment 15. The anti-B7-H4 construct of any one of embodiments 1-14, wherein the antibody moiety comprises a humanized antibody.

Embodiment 16. The anti-B7-H4 construct of any one of embodiments 1-15, wherein the B7-H4 is a human B7-H4.

Embodiment 17. A pharmaceutical composition comprising the anti-B7-H4 construct of any one of embodiments 1-16, and a pharmaceutical acceptable carrier.

Embodiment 18. An isolated nucleic acid encoding the anti-B7-H4 construct of any one of embodiments 1-16.

Embodiment 19. A vector comprising the isolated nucleic acid of embodiment 18.

Embodiment 20. An isolated host cell comprising the isolated nucleic acid of embodiment 18, or the vector of embodiment 19.

Embodiment 21. An immunoconjugate comprising the anti-B7-H4 construct of any one of embodiments 1-16, linked to a therapeutic agent or a label.

Embodiment 22. A method of producing an anti-B7-H4 construct comprising: a) culturing the isolated host cell of embodiment 20 under conditions effective to express the anti-B7-H4 construct; and b) obtaining the expressed anti-B7-H4 construct from the host cell.

Embodiment 23. A method of treating a disease or condition in an individual, comprising administering to the individual an effective mount of the anti-B7-H4 construct of any one of embodiments 1-16, or the pharmaceutical composition of embodiment 17.

Embodiment 24. The method of embodiment 23, wherein the disease or condition is a cancer.

Embodiment 25. The method of embodiment 24, wherein the cancer is a solid tumor.

Embodiment 26. The method of embodiment 24, wherein the cancer is a liquid tumor.

Embodiment 27. The method of any one of embodiments 24-26, wherein the cancer is a locally advanced or metastatic cancer.

Embodiment 28. The method of any one of embodiments 24-27, wherein the cancer is B7-H4-positive.

Embodiment 29. The method of any one of embodiments 24-28, wherein the cancer is selected from the group consisting of a lymphoma, colon cancer, breast cancer, ovarian cancer, endometrial cancer, esophageal cancer, prostate cancer, cervical cancer, renal cancer, bladder cancer, gastric cancer, non-small cell lung cancer, melanoma, and pancreatic cancer.

Embodiment 30. The method of any one of embodiments 23-29, wherein the anti-B7-H4 construct is administered parenterally into the individual.

Embodiment 31. The method of any one of embodiments 23-30, wherein the method further comprises administering a second agent.

Embodiment 32. The method of embodiment 31, wherein the second agent comprises an immunomodulatory agent.

Embodiment 33. The method of embodiment 32, wherein the immunomodulatory agent comprises an immune checkpoint inhibitor.

Embodiment 34. The method of embodiment 33, wherein the immune checkpoint inhibitor comprises an anti-PD-1 antibody.

Embodiment 35. The method of embodiment 33, wherein the immune checkpoint inhibitor is an anti-PD-L1 antibody.

Embodiment 36. The method of embodiment 32, wherein the immunomodulatory agent comprises an immune co-stimulatory agonist.

Embodiment 37. The method of embodiment 36, wherein the immune co-stimulatory agonist is an anti-4-1BB agonist.

Embodiment 38. The method of any one of embodiments 31-37, wherein the second agent is administered concurrently with the anti-B7-H4 agent.

Embodiment 39. The method of any one of embodiments 31-37, wherein the second agent is administered simultaneously with the anti-B7-H4 agent.

Embodiment 40. The method of any one of embodiments 31-37, wherein the second agent is administered sequentially with the anti-B7-H4 agent.

Embodiment 41. The method of any one of embodiments 23-40, wherein the individual is a human.

Embodiment 42. A method of modulating a cell composition, comprising contacting the cell composition with the anti-B7-H4 construct of any one of embodiments 1-16, or the pharmaceutical composition of embodiment 17.

Embodiment 43. The method of embodiment 42, wherein the cell composition comprises T cells.

### EXAMPLES

The examples below are intended to be purely exemplary of the application and should therefore not be considered to limit the application in any way. The following examples and detailed description are offered by way of illustration and not by way of limitation.

### Example 1: Generation of Anti-B7-H4 Antibodies

To generate antibodies cross-reactive against human and mouse B7-H4, CD-1/Swiss or NZB/W mice were immunized with alternating recombinant human and mouse B7-H4 Fc-tagged proteins (Sino Biological) using alhydrogel/MDP adjuvant and footpad injection on a rapid protocol with 10 injections over a course of 4 weeks. Immunized animals were bled after 3 weeks and serum titers determined for binding to human B7-H4 stably expressed on HEK-293T cells using flow cytometry (described below). Animals with the highest titer were given a final boost with recombinant protein and spleens isolated 4 weeks after the initial immunization. Hybridomas were generated by PEG fusion of splenocytes with Sp2/0-Ag14 myeloma cells. Fused cells were selected in IMDM medium containing HAT, L-Glutamine, HEPES, 10% FBS, and DCGF growth factor supplement in bulk culture for two weeks to generate hybridoma libraries.

Hybridoma library supernatants were assayed for binding to human B7-H4 expressing HEK-293T cells by flow cytometry and libraries with the highest titer were subjected to single cell sorting by FACS for clonal growth. Single cell clones were grown for two weeks in IMDM with L-Glutamine, HEPES, 10% FBS, and DCGF. Single cell hybridoma supernatants were then screened for binding to human and mouse B7-H4 protein by ELISA. 96-well hi-binding plates were coated with 0.5 μg/ml of recombinant human or mouse B7-H4 His-tagged protein (Sino Biological) in PBS and incubated overnight at 4° C. Wells were washed twice in PBS containing 0.5% Tween-20 (PBST) and blocked with PBST containing 0.5% FCS for two hours at room temperature. After washing, 50 μl of supernatant was added and incubated for 2 hours at room temperature. Supernatants were then removed and wells washed three times with PBST, followed by addition of 100 μl of horseradish peroxidase-conjugated goat anti-mouse IgG antibodies (Thermo Scientific) at an optimized dilution in PBST containing 0.5% FBS for two hours. Wells were washed three times with PBST, developed with 50 μl of TMB substrate (Thermo Scientific) for 3-5 minutes at room temperature, followed by the addition of 50 μl of stop solution (Thermo Scientific) and read at 450 nm. Hybridomas cross-reactive with both human and mouse B7-H4 were harvested, expanded, and antibodies in the supernatants were purified by protein G (HiTrap Protein G HP, GE Healthcare). Using these methods, approximately 70 hybridomas were generated.

### Example 2: In Vitro Binding Characterization of Anti-B7-H4 Monoclonal Antibodies to B7-H4 Protein and B7-H4 Expressed on the Surface of Cells

Supernatants from ELISA-positive clones or purified antibodies were further tested for their ability to bind to human and mouse B7-H4 expressed on the cell surface of HEK-283T cells by flow cytometry. HEK-293T cells were transfected with full-length human (293T-hB7-H4) or mouse B7-H4 (293T-mB7-H4) and selected for stable expression. The B7-H4-expressing 293T cells were harvested and 1×10⁵ cells per well plated in 96-well U-bottom plates and the supernatant removed. 50 μl of hybridoma supernatant or purified antibody at the specified dilution or concentration was added to each well and incubated at 4° C. for 30 minutes. Assay plates were washed 2 times with PBS and antibody binding detected using a PE-labeled goat anti-mouse antibody (Biolegend) at 1:1000 dilution in PBS for 30 minutes at 4° C. Plates were washed 2 times and analyzed on a BD LSRFortessa X-20 flow cytometer (Becton Dickinson).

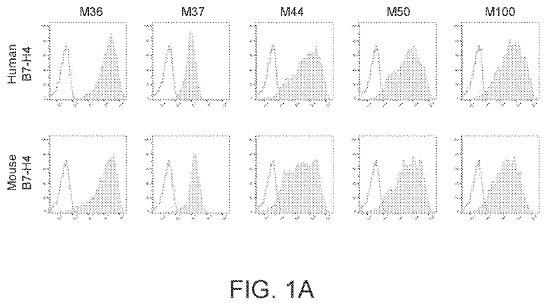

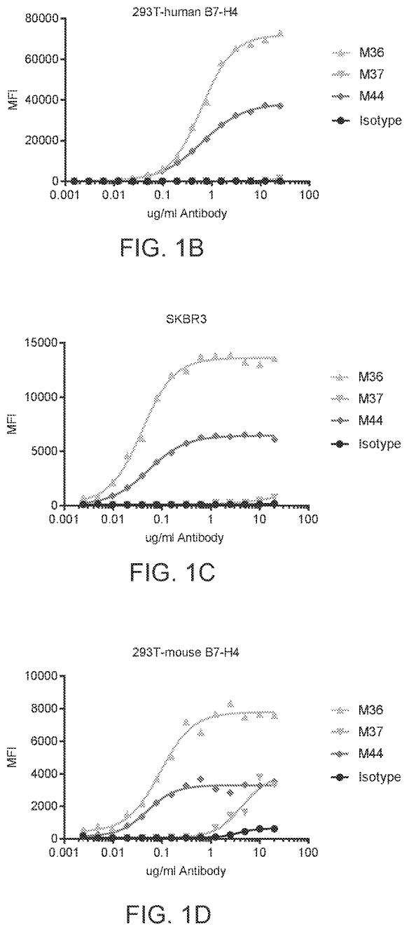

Alternatively, hybridoma supernatants or purified antibodies were tested for their ability to bind to endogenous human B7-H4 naturally expressed on the cell surface of SKBR3 cells. FIGS. 1A, 1B, 1C, and ID show that most anti-B7-H4 antibodies were cross-reactive to human and mouse B7-H4 in a dose-dependent manner.

### Example 3: In Vitro Functional Characterization of Monoclonal Antibodies

### A. Staphylococcal Enterotoxin B Stimulation of PBMCs

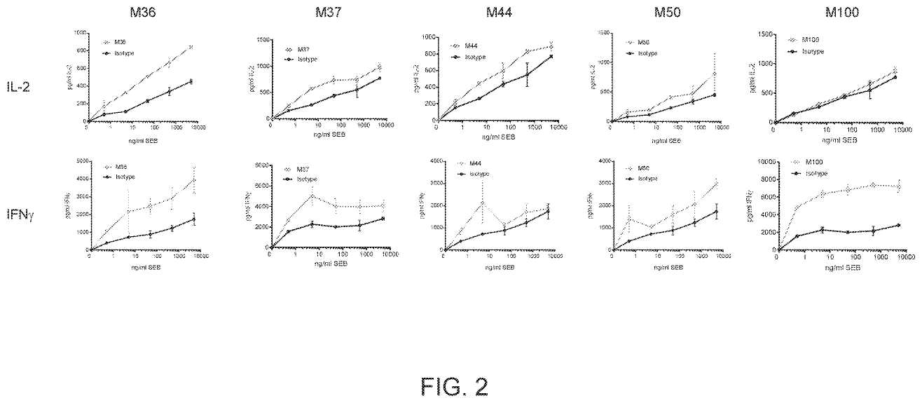

10⁵ PBMCs from healthy donors (n=4) were stimulated with serial dilutions of staphylococcal enterotoxin B (SEB, Toxin Technology) in the presence of a fixed amount of purified anti-B7-H4 antibody or isotype control antibody in solution (20 μg/ml final concentration in assay). Supernatants were collected after 72 hours for measurement of IL-2 and IFNγ levels using MSD V-plex human IL-2 or IFNγ assays (Meso Scale Discovery). FIG. 2 shows that selected B7-H4 antibodies augmented IL-2 and IFNγ production in PBMC cultures stimulated with SEB.

### B. T Cell Checkpoint Inhibition Assay

To further identify anti-B7-H4 antibodies that blocked B7-H4 T cell checkpoint function, a T cell and artificial antigen presenting cell (aAPC) co-culture assay was devised. Primary human T cells were isolated from peripheral blood mononuclear cells (PBMC) of healthy human donors using the Pan T Cell Isolation Kit (Miltenyi) according to the manufacturer's instructions. Isolated T cells were labeled in a 5 μM CFSE solution (Molecular Probes) for 12 min at 37° C., washed, and resuspended in RPMI containing 10% FBS. 1×10⁵ CFSE-labeled T cells were then cultured with 1×10⁵ cells aAPCs in the presence of 10 μg/ml antibody or a dose titration of antibody in a final volume of 200 μl RPMI, aAPCs are HEK293T cells transfected and selected to stably express the scFv of the human anti-CD3 OKT3 clone, as well as full length human B7-H4 on the cell surface. Prior to their addition to T cells, aAPCs were treated with 50 μg/ml Mitomycin C for 1 hour at 37° C. and washed 3 times with RPMI medium. T cell, aAPC, and antibody co-cultures were incubated for 72 hours at 37° C., after which the supernatants were harvested by centrifugation and measured for IFNγ levels using MSD V-plex assays (Meso Scale Discovery). T cell proliferation within the co-culture was simultaneously assessed by staining cells with fluorophore-conjugated anti-CD3, anti-CD4, and anti-CD8 antibodies (Biolegend) and Live/Dead Fixable dead cell stain (Molecular Probes), and running on a BD LSRFortessa X-20 flow cytometer. FACS data was analyzed using FlowJo software. Data for single dose concentrations of antibody are shown as the fold change in proliferation compared against control mouse IgG2a antibody, and as IFNγ production levels. The effects of antibody dose titrations are represented as the percent increase in proliferating T cells over control mouse IgG2a antibody vs. antibody concentration.

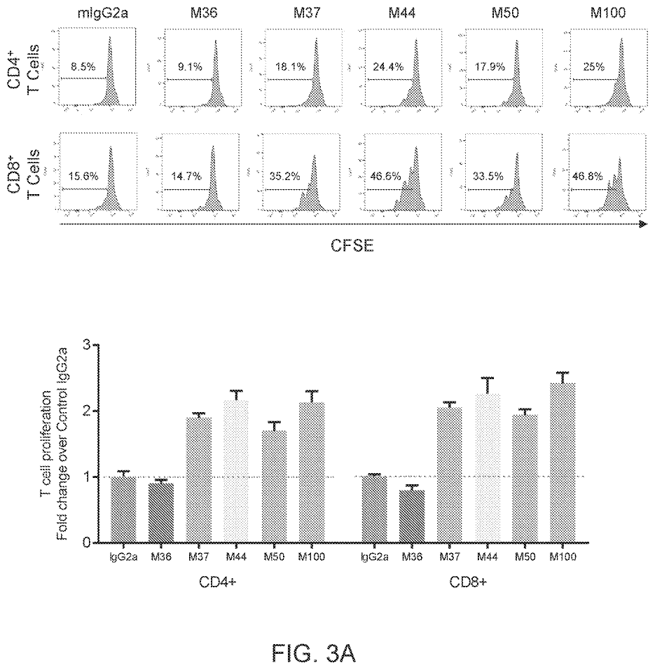

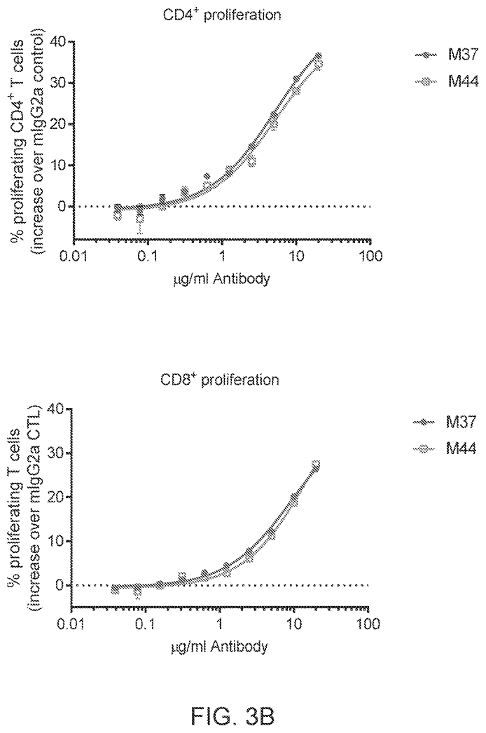

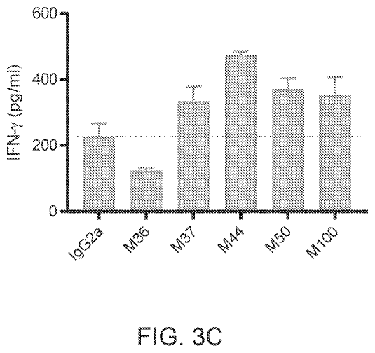

FIG. 3A shows CFSE proliferation histograms of CD4+ and CD8+ T cells in the presence of 10 μg/ml of control mIgG2a antibody or various anti-B7-H4 antibodies, as well as the quantitative fold change in T cell proliferation for each antibody compared to control antibody. FIG. 3B shows dose dependent effects of the anti-B7-H4 antibodies M37 and M44 on B7-H4 checkpoint inhibition as measured by T cell proliferation. IFNγ production levels in the presence of anti-B7-H4 antibodies are shown in FIG. 3C.

### Example 4: Anti-Tumor Activity of Anti-B7-H4 and Anti-PD-1 Antibodies in an Animal Tumor Model

Approximately seven to eight week old female C57BL/6 mice with an average weight of 20 grams were obtained from Taconic Laboratory. E.G7-OVA murine T cell lymphoma cells syngeneic to the C57BL/6 mouse strain were cultured in RPMI medium supplemented with 10% FBS at 37° C. with 5% CO₂. 1×106 log-phase E.G7-OVA cells were injected subcutaneously in a 100 ml volume in the right flank of each mouse. Mice were shaved in the area used for tumor implantation using electric clippers beforehand.

Tumor growth was measured twice a week following implantation. Tumor length and width were measured using electronic calipers and tumor volume determined using the formula Volume (mm³)=0.5×Length×Width2 where length is the longer dimension. Mice were weighed once a week to monitor general health and to also calculate actual mg/kg dose delivery per mouse.

In a tumor prevention setting, animals were randomized into treatment groups of 10 mice per group on day 5, prior to the emergence of palpable and measurable tumors. Antibody treatments were administered every 4 days for a total of four doses, starting on day 5 post-inoculation at a 5 mg/kg dose.

For anti-B7-H4 treatment, the antibodies included M36, M37, M44, M50, and M100. For control, mice were administered mouse isotype IgG1 (Leinco P382). For relative comparison, one mouse cohort was also administered anti-mouse PD-1 antibody (Leinco P372). All antibodies were prepared in PBS.

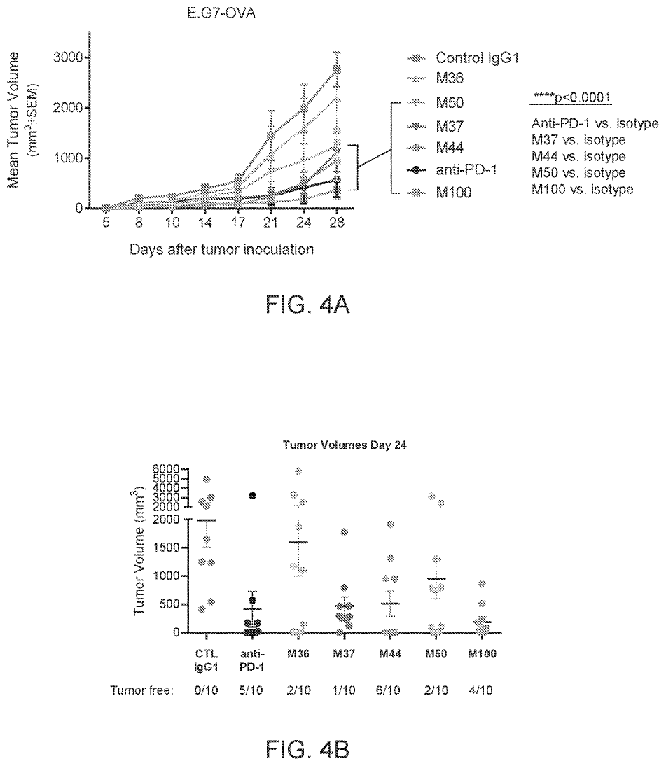

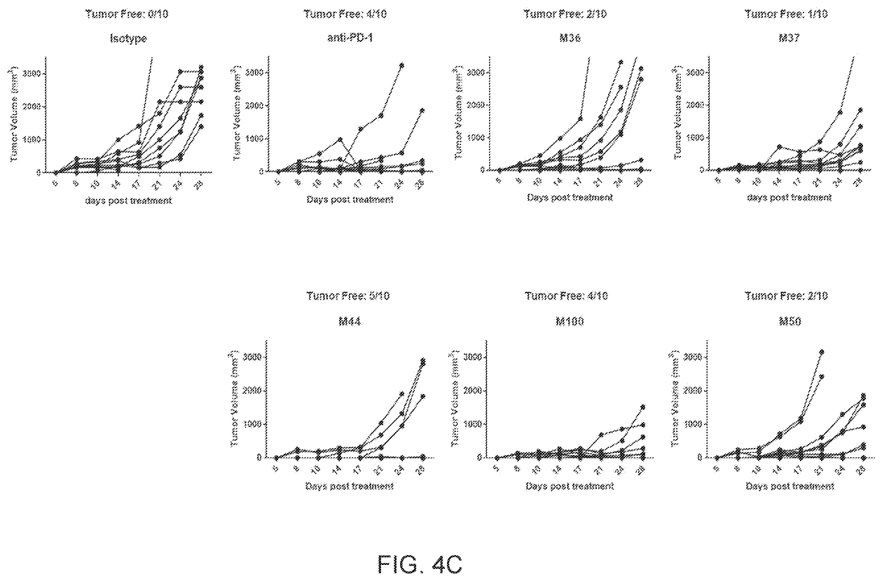

Tumors continued to be monitored twice a week until tumor volumes exceeded 2000 mm³, at which point animals were euthanized. As shown in FIG. 4A, the mean tumor volumes of mice treated with M37, M44, M50, M100, and anti-PD-1 (p<0.0001 at day 28) were significantly reduced compared to isotype control treatment. Individual tumor volumes at day 24 are shown in FIG. 4B and demonstrate that treatment groups with significant anti-tumor effect have many tumor-free animals. FIG. 4C shows the individual tumor growth curves over time for each antibody treatment.

### Example 5: Anti-Tumor Activity of Anti-B7-H4 in Combination with Anti-PD-1 Antibodies in Animal Tumor Models

Approximately seven to eight week old female C57BL/6J mice with an average weight of 20 grams were obtained from Taconic Laboratory. MC38 murine colon adenocarcinoma cells syngeneic to the C57BL/6J mouse strain were cultured in DMEM medium supplemented with 10% FBS at 37° C. with 5% CO2. 1×106 log-phase and subconfluent MC38 cells were injected subcutaneously in a 100 μl volume in the right flank of each mouse. Mice were shaved in the area used for tumor implantation using electric clippers beforehand.

Tumor growth was measured twice a week following implantation. Tumor length and width were measured using electronic calipers and tumor volume determined using the formula Volume (mm³)=0.5×Length×Width2 where length is the longer dimension. Mice were weighed once a week to monitor general health and to also calculate actual mg/kg dose delivery per mouse.

Treatment was started once the tumor size averaged 70 mm³ (20-85 mm³). On the day of treatment initiation, all tumors were measured and any outliers removed, and the remaining mice randomized into various treatment groups of 10 mice per group with equivalent mean tumor size. Antibody treatments were initiated on day 11 and administered every 3 days for a total of four doses at the dosing concentrations specified below.

For anti-B7-H4 treatment, mice were treated with 10 mg/kg M44. For control, mice were administered 10 mg/kg mouse isotype IgG1 (Leinco P382) and/or 0.5 mg/kg rat IgG2a isotype (Leinco R1367). A rat antibody against mouse PD-1 (Leinco P372) was used at 0.5 mg/kg, and a rat antibody against mouse 4-1BB (Bio X Cell BE0239) was used at 1 mg/kg. All antibodies were prepared in PBS.

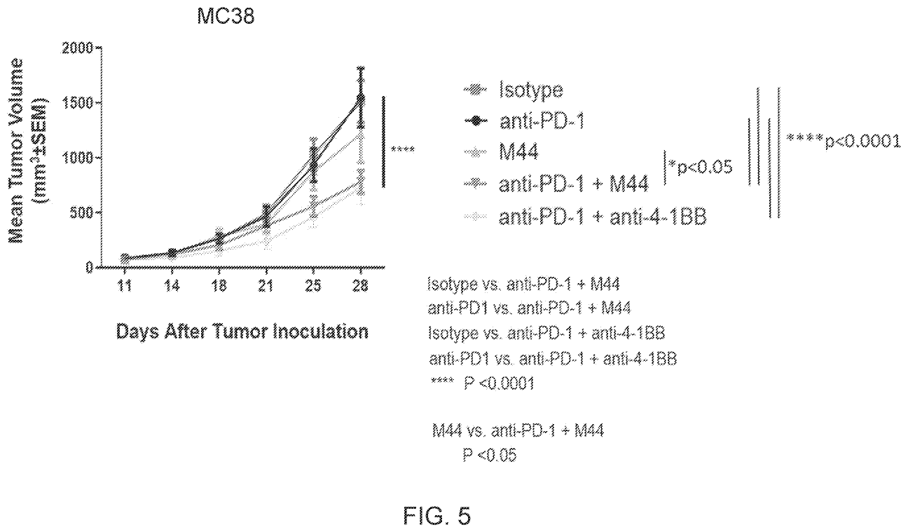

Tumors continued to be monitored twice a week until tumor volumes exceeded 2000 mm³, at which point animals were euthanized. As shown in FIG. 5, treatment with anti-PD-1 as monotherapy had no effect compared with control treatment, whereas M44 monotherapy had only a mild, statistically insignificant anti-tumor effect at day 28. In contrast, combination treatment with both anti-PD-1 and M44 provided a significant anti-tumor effect compared to isotype control (p<0.0001), anti-PD-1 alone (p<0.0001), or M44 alone (p<0.05). Treatment with anti-PD-1 and anti-4-1BB in combination was included as a positive comparator as it is known to be efficacious in this model. The results from the combination of anti-PD-1 with M44 were similar to those from the combination of anti-PD-1 with anti-4-1BB.

Similar combination therapy experiments were performed using BALB/c mice implanted with A20 murine B cell lymphoma cells. A20 cells syngeneic to the BALB/c mouse strain were cultured in RPMI medium supplemented with 10% FBS at 37° C. with 5% CO2. 3×106 log-phase A20 cells were injected subcutaneously in a 100 □l volume in the right flank of each mouse. Treatment was started once the tumor size averaged 50 mm³. Antibody treatments were initiated on day 6 and administered every 4 days for a total of four doses at 5 mg/kg for all antibodies.

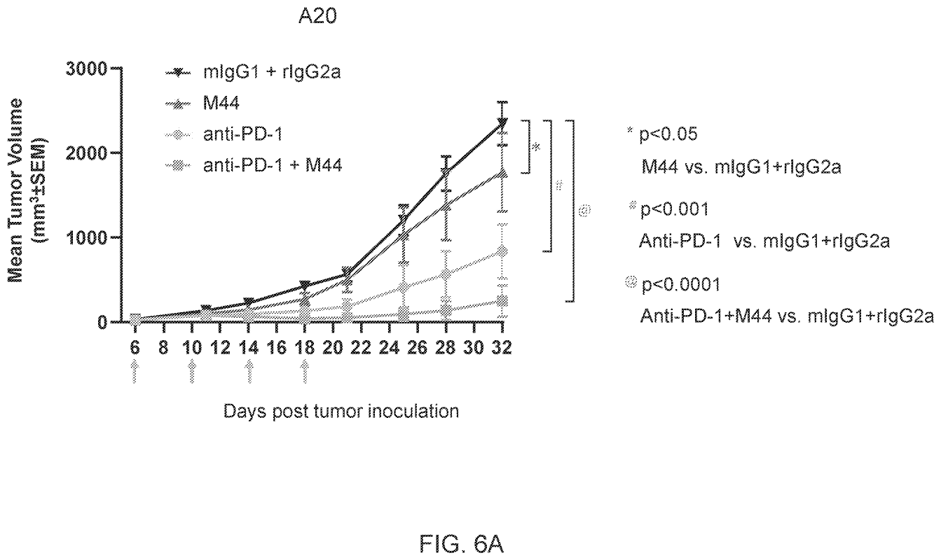

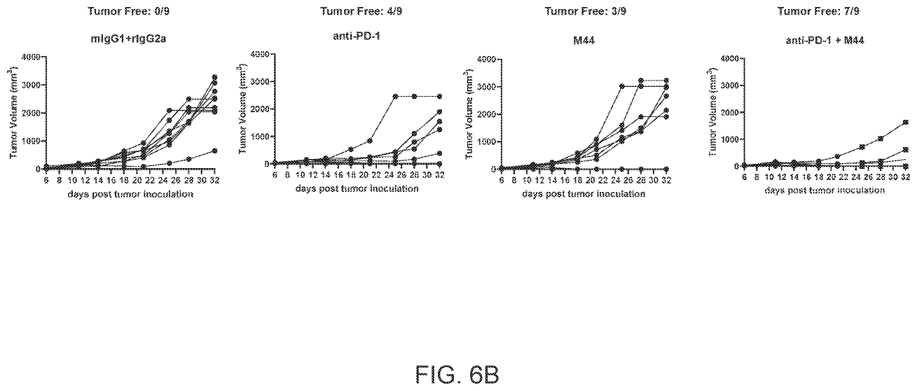

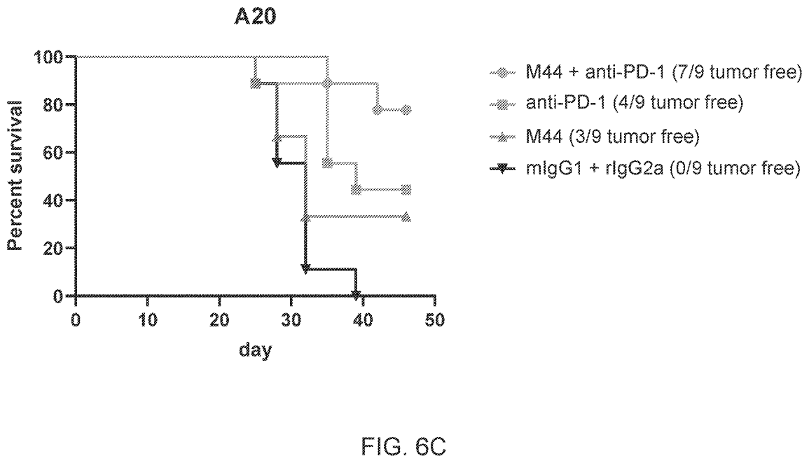

FIG. 6A shows that both M44 (p<0.05) and anti-PD-1 (p<0.001) have anti-tumor efficacy as monotherapies compared to isotype control treatment, but that the combination of M44 with anti-PD-1 further enhances anti-tumor efficacy compared to control or either monotherapy alone. FIG. 6B shows the individual tumor growth curves and the number of tumor free animals for each treatment group. FIG. 6C shows that combination therapy also resulted in better survival compared to either monotherapy.

SEQUENCE TABLE

| SEQ | | Nucleotide or Amino acid sequence (CDR sequences are |
|---|---|---|
| ID NO | Description | underlined and bold) |
| 1 | M44 VH-CDR1 | DFTIH |
| 2 | M44 VH-CDR2 | WIYPGSDNTKYNDNFKV |
| 3 | M44 VH-CDR3 | SGGNLFAY |
| 4 | M44 VL-CDR1 | SASSSVSYMH |
| 5 | M44 VL-CDR2 | TTSNLAS |
| 6 | M44 VL-CDR3 | QQRSSYPLT |
| 7 | M44 VH | QVQLQQSGPELVRPGASVKLSCRASGYTFTDFTIHWVKQSPGQ |
| | | GLEWIGWIYPGSDNTKYNDNFKVKATLTADKSSSTAYMQLSS |
| | | LTSEDSAVYFCARSGGNLFAYWGQGTLVTVST |
| 8 | M44 VL | QIVLTQSPAIMSASPGEKVTITCSASSSVSYMHWFQQRPGTSPK |
| | | LWIYTTSNLASGVPARFSGSGSGTSYSLTISRMEAEDAATYYCQ |
| | | QRSSYPLTFGAGTKLELK |
| 9 | Amino acid | QVQLQQSGPELVRPGASVKLSCRASGYTFTDFTIHWVKQSPGQ |
| | sequence of | GLEWIGWIYPGSDNTKYNDNFKVKATLTADKSSSTAYMQLSS |
| | heavy chain of | LTSEDSAVYFCARSGGNLFAYWGQGTLVTVSTAKTTPPSVYPL |
| | M44 | APGSAAQTNSMVTLGCLVKGYFPEPVTVTWNSGSLSSGVHTFP |
| | | AVLQSDLYTLSSSVTVPSSTWPSETVTCNVAHPASSTKVDKKIV |
| | | PRDCGCKPCICTVPEVSSVFIFPPKPKDVLTITLTPKVTCVVVDIS |
| | | KDDPEVQFSWFVDDVEVHTAQTQPREEQFNSTFRSVSELPIMH |
| | | QDWLNGKEFKCRVNSAAFPAPIEKTISKTKGRPKAPQVYTIPPP |
| | | KEQMAKDKVSLTCMITDFFPEDITVEWQWNGQPAENYKNTQPI |
| | | MDTDGSYFIYSKLNVQKSNWEAGNTFTCSVLHEGLHNHHTEK |
| | | SLSHSPGK |
| 10 | Amino acid | QIVLTQSPAIMSASPGEKVTITCSASSSVSYMHWFQQRPGTSPK |
| | sequence of | LWIYTTSNLASGVPARFSGSGSGTSYSLTISRMEAEDAATYYCQ |
| | light chain of | QRSSYPLTFGAGTKLELKRADAAPTVSIFPPSSEQLTSGGASVV |
| | M44 | CFLNNFYPKDINVKWKIDGSERQNGVLNSWTDQDSKDSTYSM |
| | | SSTLTLTKDEYERHNSYTCEATHKTSTSPIVKSFNRNEC |
| 11 | M37 VH-CDR1 | NYWMH |
| 12 | M37 VH-CDR2 | YINPSSGYTEYSQKFKD |
| 13 | M37 VH-CDR3 | PYGSFAY |
| 14 | M37 VL-CDR1 | RASQSISNNLH |
| 15 | M37 VL-CDR2 | FASQSIS |
| 16 | M37 VL-CDR3 | QQSNSWPYT |
| 17 | M37 VH | QVQLQQSGAELAKPGASVRMSCKASGYTFTNYWMHWVKQRP |
| | | GQGLEWIGYINPSSGYTEYSQKFKDKATLTADKSSSTAYMQL |
| | | TSLTSEDSAVYYCAGPYGSFAYWGQGTLVTVSA |
| 18 | M37 VL | DIKMTQSPATLSVTPGDSVSLSCRASQSISNNLHWYQQKSHESP |
| | | RLLIKFASQSISGIPSRFSGSGSGTDFTLSINSVETEDFGMYFCQQ |
| | | SNSWPYTFGGGTKLEIK |
| 19 | Amino acid | QVQLQQSGAELAKPGASVRMSCKASGYTFTNYWMHWVKQRP |
| | sequence of | GQGLEWIGYINPSSGYTEYSQKFKDKATLTADKSSSTAYMQL |
| | heavy chain of | TSLTSEDSAVYYCAGPYGSFAYWGQGTLVTVSAAKTTPPSVYP |
| | M37 | LAPGCGDTTGSSVTLGCLVKGYFPESVTVTWNSGSLSSSVHTFP |
| | | ALLQSGLYTMSSSVTVPSSTWPSQTVTCSVAHPASSTTVDKKLE |
| | | PSGPISTINPCPPCKECHKCPAPNLEGGPSVFIFPPNIKDVLMISLT |
| | | PKVTCVVVDVSEDDPDVQISWFVNNVEVHTAQTQTHREDYNS |
| | | TIRVVSTLPIQHQDWMSGKEFKCKVNNKDLPSPIERTISKIKGLV |
| | | RAPQVYILPPPAEQLSRKDVSLTCLVVGFNPGDISVEWTSNGHT |
| | | EENYKDTAPVLDSDGSYFIYSKLNMKTSKWEKTDSFSCNVRHE |
| | | GLKNYYLKKTISRSPGK |
| 20 | Amino acid | DIKMTQSPATLSVTPGDSVSLSCRASQSISNNLHWYQQKSHESP |
| | sequence of | RLLIKFASQSISGIPSRFSGSGSGTDFTLSINSVETEDFGMYFCQQ |
| | light chain of | SNSWPYTFGGGTKLEIKRADAAPTVSIFPPSSEQLTSGGASVVC |
| | M37 | FLNNFYPKDINVKWKIDGSERQNGVLNSWTDQDSKDSTYSMSS |
| | | TLTLTKDEYERHNSYTCEATHKTSTSPIVKSFNRNEC |
| 21 | M36 VH-CDR1 | GYWIE |
| 22 | M36 VH-CDR2 | EILPGSGSTNYNEKFKG |
| 23 | M36 VH-CDR3 | RTWLYAMDY |
| 24 | M36 VL-CDR1 | KASQSVGNNVV |
| 25 | M36 VL-CDR2 | YASNRSP |
| 26 | M36 VL-CDR3 | QQHYRSFT |
| 27 | M36 VH | EVQLKESGAELMKPGASVKLSCKATGYTFTGYWIEWVKQRPG |
| | | HGLEWIGEILPGSGSTNYNEKFKGKATFTADTSSNTAYMQLSS |
| | | LTTEDSVIYYCARRTWLYAMDYWGQGTSVTVSS |
| 28 | M36 VL | SIVMTQNPKFLPVSAGDRVTMTCKASQSVGNNVVWYQQKPG |
| | | QSPKLLIYYASNRSPGVPDRFTGSGSGTDFTFTISSVQVEDLAVY |
| | | FCQQHYRSFTFGTGTKLEIK |
| 29 | Amino acid | EVQLKESGAELMKPGASVKLSCKATGYTFTGYWIEWVKQRPG |
| | sequence of | HGLEWIGEILPGSGSTNYNEKFKGKATFTADTSSNTAYMQLSS |
| | heavy chain of | LTTEDSVIYYCARRTWLYAMDYWGQGTSVTVSSAKTTAPSVY |
| | M36 | PLAPVCGGTTGSSVTLGCLVKGYFPEPVTLTWNSGSLSSGVHTF |
| | | PALLQSGLYTLSSSVTVTSNTWPSQTITCNVAHPASSTKVDKKI |
| | | EPRVPITQNPCPPLKECPPCAAPDLLGGPSVFIFPPKIKDVLMISL |
| | | SPMVTCVVVDVSEDDPDVQISWFVNNVEVHTAQTQTHREDYN |
| | | STLRVVSALPIQHQDWMSGKEFKCKVNNRALPSPIEKTISKPRG |
| | | PVRAPQVYVLPPPAEEMTKKEFSLTCMITGFLPAEIAVDWTSNG |
| | | RTEQNYKNTATVLDSDGSYFMYSKLRVQKSTWERGSLFACSV |
| | | VHEGIHNHLTTKTISHSPGK |
| 30 | Amino acid | SIVMTQNPKFLPVSAGDRVTMTCKASQSVGNNVVWYQQKPG |
| | sequence of | QSPKLLIYYASNRSPGVPDRFTGSGSGTDFTFTISSVQVEDLAVY |
| | light chain of | FCQQHYRSFTFGTGTKLEIKRADAAPTVSIFPPSSEQLTSGGAS |
| | M36 | VVCFLNNFYPKDINVKWKIDGSERQNGVLNSWTDQDSKDSTY |
| | | SMSSTLTLTKDEYERHNSYTCEATHKTSTSPIVKSFNRNEC |

Leading sequences

| | | |
|---|---|---|
| 31 | M44 heavy | MEWSWVMLFLLSGTAGVHC |
| | chain leading | |
| | sequence | |
| 32 | M44 light chain | MDFQVQIFSFLLISASVIMSRG |
| | leading | |
| | sequence | |
| 33 | M37 heavy | MKWSWVCILFLLSVTAGVHS |
| | chain leading | |
| | sequence | |
| 34 | M37 light chain | MGWSCIILFLVATATGVHS |
| | leading | |
| | sequence | |
| 35 | M36 heavy | MGWSLILLFLVAVATRVLS |
| | chain leading | |
| | sequence | |
| 36 | M36 light chain | METQTQVFIFLLLCVSGAHG |
| | leading | |
| | sequence | |

Exemplary linkers

| | | |
|---|---|---|
| 37 | Linker | (G)ₙ, n >= 1 |
| 38 | Linker | (GS)ₙ, 20 >= n >= 1 |
| 39 | Linker | (GSGGS)ₙ, 8 >= n >= 1 |
| 40 | Linker | (GGGGS)ₙ, 8 >= n >=1 |
| 41 | Linker | (GGGS)ₙ, 8 >= n >=1 |
| 42 | Linker | (GGGGS)₃ |
| 43 | Linker | (GGGGS)₆ |
| 44 | Linker | (GSTSGSGKPGSGEGS)ₙ |
| | | 3 >= n >= 1 |

The invention claimed is:

1. An anti-B7-H4 construct comprising an antibody moiety comprising a heavy chain variable region (V_{H}) and a light chain variable region (V_{L}), wherein the antibody moiety competes for a binding epitope of B7-H4 with an antibody or antibody fragment comprising a second heavy variable region (V_{H-2}) and a second light chain variable region (V_{L-2}), wherein:
a) the V_{H-2} comprises the HC-CDR1 comprising the amino acid sequence of SEQ ID NO: 1, the HC-CDR2 comprising the amino acid sequence of SEQ ID NO: 2, and the HC-CDR3 comprising the amino acid sequence of SEQ ID NO: 3, and the V_{L-2} comprises the LC-CDR1 comprising the amino acid sequence of SEQ ID NO: 4, the LC-CDR2 comprising the amino acid sequence of SEQ ID NO: 5, and the LC-CDR3 comprising the amino acid sequence of SEQ ID NO: 6;
b) the V_{H-2} comprises the HC-CDR1 comprising the amino acid sequence of SEQ ID NO: 11, the HC-CDR2 comprising the amino acid sequence of SEQ ID NO: 12, and the HC-CDR3 comprising the amino acid sequence of SEQ ID NO: 13, and the V_{L-2} comprises the LC-CDR1 comprising the amino acid sequence of SEQ ID NO: 14, the LC-CDR2 comprising the amino acid sequence of SEQ ID NO: 15, and the LC-CDR3 comprising the amino acid sequence of SEQ ID NO: 16; or
c) the V_{H-2} comprises the HC-CDR1 comprising the amino acid sequence of SEQ ID NO: 21, the HC-CDR2 comprising the amino acid sequence of SEQ ID NO: 22, and the HC-CDR3 comprising the amino acid sequence of SEQ ID NO: 23, and the V_{L-2} comprises the LC-CDR1 comprising the amino acid sequence of SEQ ID NO: 24, the LC-CDR2 comprising the amino acid sequence of SEQ ID NO: 25, and the LC-CDR3 comprising the amino acid sequence of SEQ ID NO: 26.

2. The anti-B7-H4 construct of claim 1, wherein:
a) the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO. 3, or a variant thereof comprising up to 3, 2, or 1 amino acid substitutions in the HC-CDRs, and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 3, 2, or 1 amino acid substitutions in the LC-CDRs;
b) the V_{H} comprises i) the HC-CDR1 comprising the amino acid sequence of SEQ ID NO: 11, ii) the HC-CDR2 comprising the amino acid sequence of SEQ ID NO: 12, and iii) the HC-CDR3 comprising the amino acid sequence of SEQ ID NO: 13, or a variant thereof comprising up to 3, 2, or 1 amino acid substitutions in the HC-CDRs, and the V_{L} comprises i) the LC-CDR1 comprising the amino acid sequence of SEQ ID NO: 14, ii) the LC-CDR2 comprising the amino acid sequence of SEQ ID NO: 15, and iii) the LC-CDR3 comprising the amino acid sequence of SEQ ID NO: 16, or a variant thereof comprising up to 3, 2, or 1 amino acid substitutions in the LC-CDRs; or
c) the V_{H} comprises i) the HC-CDR1 comprising the amino acid sequence of SEQ ID NO: 21, ii) the HC-CDR2 comprising the amino acid sequence of SEQ ID NO: 22, and iii) the HC-CDR3 comprising the amino acid sequence of SEQ ID NO: 23, or a variant thereof comprising up to 3, 2, or 1 amino acid substitutions in the HC-CDRs, and the V_{L} comprises i) the LC-CDR1 comprising the amino acid sequence of SEQ ID NO: 24, ii) the LC-CDR2 comprising the amino acid sequence of SEQ ID NO: 25, and iii) the LC-CDR3 comprising the amino acid sequence of SEQ ID NO: 26, or a variant thereof comprising up to 3, 2, or 1 amino acid substitutions in the LC-CDRs.

3. The anti-B7-H4 construct of claim 2, wherein the V_{H} comprises i) the HC-CDR1 comprising the amino acid sequence of SEQ ID NO: 1, ii) the HC-CDR2 comprising the amino acid sequence of SEQ ID NO: 2, and iii) the HC-CDR3 comprising the amino acid sequence of SEQ ID NO: 3, or a variant thereof comprising up to 3, 2, or 1 amino acid substitutions in the HC-CDRs; and the V_{L} comprises i) the LC-CDR1 comprising the amino acid sequence of SEQ ID NO: 4, ii) the LC-CDR2 comprising the amino acid sequence of SEQ ID NO: 5, and iii) the LC-CDR3 comprising the amino acid sequence of SEQ ID NO: 6, or a variant thereof comprising up to 3, 2, or 1 amino acid substitutions in the LC-CDRs.

4. The anti-B7-H4 construct of claim 2, wherein the V_{H} comprises i) the HC-CDR1 comprising the amino acid sequence of SEQ ID NO: 11, ii) the HC-CDR2 comprising the amino acid sequence of SEQ ID NO: 12, and iii) the HC-CDR3 comprising the amino acid sequence of SEQ ID NO: 13, or a variant thereof comprising up to 3, 2, or 1 amino acid substitutions in the HC-CDRs; and the V_{L} comprises i) the LC-CDR1 comprising the amino acid sequence of SEQ ID NO: 14, ii) the LC-CDR2 comprising the amino acid sequence of SEQ ID NO: 15, and iii) the LC-CDR3 comprising the amino acid sequence of SEQ ID NO: 16, or a variant thereof comprising up to 3, 2, or 1 amino acid substitutions in the LC-CDRs.

5. The anti-B7-H4 construct of claim 2, wherein the V_{H} comprises i) the HC-CDR1 comprising the amino acid sequence of SEQ ID NO: 21, ii) the HC-CDR2 comprising the amino acid sequence of SEQ ID NO: 22, and iii) the HC-CDR3 comprising the amino acid sequence of SEQ ID NO: 23, or a variant thereof comprising up to 3, 2, or 1 amino acid substitutions in the HC-CDRs; and the V_{L} comprises i) the LC-CDR1 comprising the amino acid sequence of SEQ ID NO: 24, ii) the LC-CDR2 comprising the amino acid sequence of SEQ ID NO: 25, and iii) the LC-CDR3 comprising the amino acid sequence of SEQ ID NO: 26, or a variant thereof comprising up to 3, 2, or 1 amino acid substitutions in the LC-CDRs.

6. An anti-B7-H4 construct comprising an antibody moiety that specifically binds to B7-H4, comprising:
a) a heavy chain variable region (V_{H}) comprising a HC-CDR1, a HC-CDR2, and a HC-CDR3, respectively comprising the amino acid sequences of a CDR1, a CDR2, and a CDR3 within a V_{H} chain region having the sequence set forth in any one of SEQ ID NOs: 7, 17 and 27; and
b) a light chain variable region (V_{L}) comprising a LC-CDR1, a LC-CDR2, and a LC-CDR3, respectively comprising the amino acid sequences of a CDR1, a CDR2, and a CDR3 within a V_{L} chain region having the sequence set forth in any one of SEQ ID NOs: 8, 18, and 28.

7. The anti-B7-H4 construct of any one of claims 1-6, wherein the V_{H} comprises an amino acid sequence of any one of SEQ ID NOs: 7, 17, and 27, or a variant comprising an amino acid sequence having at least about 80% sequence identity; and/or wherein the V_{L} comprises an amino acid sequence of any one of SEQ ID NOs: 8, 18, and 28, or a variant comprising an amino acid sequence having at least about 80% sequence identity.

8. The anti-B7-H4 construct of claim 7, wherein:
1) the V_{H} comprises an amino acid sequence of SEQ ID NO: 7, or a variant comprising an amino acid sequence having at least about 80% sequence identity; and wherein the V_{L} comprises an amino acid sequence of SEQ ID NO: 8, or a variant comprising an amino acid sequence having at least about 80% sequence identity;
2) the V_{H} comprises an amino acid sequence of SEQ ID NO: 17, or a variant comprising an amino acid sequence having at least about 80% sequence identity; and wherein the V_{L} comprises an amino acid sequence of SEQ ID NO: 18, or a variant comprising an amino acid sequence having at least about 80% sequence identity; or
3) the V_{H} comprises an amino acid sequence of SEQ ID NO: 27, or a variant comprising an amino acid sequence having at least about 80% sequence identity; and wherein the V_{L} comprises an amino acid sequence of SEQ ID NO: 28, or a variant comprising an amino acid sequence having at least about 80% sequence identity.

9. The anti-B7-H4 construct of any one of claims 1-8, wherein the construct is an antibody or antigen-binding fragment thereof selected from the group consisting of a full-length antibody, a bispecific antibody, a single-chain Fv (scFv) fragment, a Fab fragment, a Fab′ fragment, a F(ab′)2, an Fv fragment, a disulfide stabilized Fv fragment (dsFv), a (dsFv)2, a V_{H}H, a Fv-Fc fusion, a scFv-Fc fusion, a scFv-Fv fusion, a diabody, a tribody, and a tetrabody.

10. The anti-B7-H4 construct of claim 9, wherein the construct comprises a full-length antibody.

11. The anti-B7-H4 construct of any one of claims 1-10, wherein the antibody moiety has an Fc fragment of an immunoglobulin selected from the group consisting of IgG, IgA, IgD, IgE, IgM, and combinations and hybrids thereof.

12. The anti-B7-H4 construct of claim 11, wherein the antibody moiety has an Fc fragment of an immunoglobulin selected from the group consisting of IgG1, IgG2, IgG3, IgG4, and combinations and hybrids thereof.

13. The anti-B7-H4 construct of claim 11 or claim 12, wherein the Fc fragment has a reduced effector function as compared to the corresponding wildtype Fc fragment.

14. The anti-B7-H4 construct of claim 11 or claim 12, wherein the Fc fragment has an enhanced effector function as compared to the corresponding wildtype Fc fragment.

15. The anti-B7-H4 construct of any one of claims 1-14, wherein the antibody moiety comprises a humanized antibody.

16. The anti-B7-H4 construct of any one of claims 1-15, wherein the B7-H4 is a human B7-H4.

17. A pharmaceutical composition comprising the anti-B7-H4 construct of any one of claims 1-16, and a pharmaceutical acceptable carrier.

18. An isolated nucleic acid encoding the anti-B7-H4 construct of any one of claims 1-16.

19. A vector comprising the isolated nucleic acid of claim 18.

20. An isolated host cell comprising the isolated nucleic acid of claim 18, or the vector of claim 19.

21. An immunoconjugate comprising the anti-B7-H4 construct of any one of claims 1-16, linked to a therapeutic agent or a label.

22. A method of producing an anti-B7-H4 construct comprising:
a) culturing the isolated host cell of claim 20 under conditions effective to express the anti-B7-H4 construct; and
b) obtaining the expressed anti-B7-H4 construct from the host cell.

23. A method of treating a disease or condition in an individual, comprising administering to the individual an effective mount of the anti-B7-H4 construct of any one of claims 1-16, or the pharmaceutical composition of claim 17.

24. The method of claim 23, wherein the disease or condition is a cancer.

25. The method of claim 24, wherein the cancer is a solid tumor.

26. The method of claim 24, wherein the cancer is a liquid tumor.

27. The method of any one of claims 24-26, wherein the cancer is a locally advanced or metastatic cancer.

28. The method of any one of claims 24-27, wherein the cancer is B7-H4-positive.

29. The method of any one of claims 24-28, wherein the cancer is selected from the group consisting of a lymphoma, colon cancer, breast cancer, ovarian cancer, endometrial cancer, esophageal cancer, prostate cancer, cervical cancer, renal cancer, bladder cancer, gastric cancer, non-small cell lung cancer, melanoma, and pancreatic cancer.

30. The method of any one of claims 23-29, wherein the anti-B7-H4 construct is administered parenterally into the individual.

31. The method of any one of claims 23-30, wherein the method further comprises administering a second agent.

32. The method of claim 31, wherein the second agent comprises an immunomodulatory agent.

33. The method of claim 32, wherein the immunomodulatory agent comprises an immune checkpoint inhibitor.

34. The method of claim 33, wherein the immune checkpoint inhibitor comprises an anti-PD-1 antibody.

35. The method of claim 33, wherein the immune checkpoint inhibitor is an anti-PD-L1 antibody.

36. The method of claim 32, wherein the immunomodulatory agent comprises an immune co-stimulatory agonist.

37. The method of claim 36, wherein the immune co-stimulatory agonist is an anti-4-1BB agonist.

38. The method of any one of claims 31-37, wherein the second agent is administered concurrently with the anti-B7-H4 agent.

39. The method of any one of claims 31-37, wherein the second agent is administered simultaneously with the anti-B7-H4 agent.

40. The method of any one of claims 31-37, wherein the second agent is administered sequentially with the anti-B7-H4 agent.

41. The method of any one of claims 23-40, wherein the individual is a human.

42. A method of modulating a cell composition, comprising contacting the cell composition with the anti-B7-H4 construct of any one of claims 1-16, or the pharmaceutical composition of claim 17.

43. The method of claim 42, wherein the cell composition comprises T cells.


SEQUENCE LISTING (44 sequences)

SEQ ID NO: 1 - protein, 5 aa, Artificial Sequence
  Synthetic Construct
DFTIH

SEQ ID NO: 2 - protein, 17 aa, Artificial Sequence
  Synthetic Construct
WIYPGSDNTKYNDNFKV

SEQ ID NO: 3 - protein, 8 aa, Artificial Sequence
  Synthetic Construct
SGGNLFAY

SEQ ID NO: 4 - protein, 10 aa, Artificial Sequence
  Synthetic Construct
SASSSVSYMH

SEQ ID NO: 5 - protein, 7 aa, Artificial Sequence
  Synthetic Construct
TTSNLAS

SEQ ID NO: 6 - protein, 9 aa, Artificial Sequence
  Synthetic Construct
QQRSSYPLT

SEQ ID NO: 7 - protein, 117 aa, Artificial Sequence
  Synthetic Construct
QVQLQQSGPELVRPGASVKLSCRASGYTFTDFTIHWVKQSPGQGLEWIGWIYPGSDNTKYNDNFKVKATLTADKSSSTAYMQLSSLTSEDSAVYFCARSGGNLFAYWGQGTLVTVST

SEQ ID NO: 8 - protein, 106 aa, Artificial Sequence
  Synthetic Construct
QIVLTQSPAIMSASPGEKVTITCSASSSVSYMHWFQQRPGTSPKLWIYTTSNLASGVPARFSGSGSGTSYSLTISRMEAEDAATYYCQQRSSYPLTFGAGTKLELK

SEQ ID NO: 9 - protein, 441 aa, Artificial Sequence
  Synthetic Construct
QVQLQQSGPELVRPGASVKLSCRASGYTFTDFTIHWVKQSPGQGLEWIGWIYPGSDNTKYNDNFKVKATLTADKSSSTAYMQLSSLTSEDSAVYFCARSGGNLFAYWGQGTLVTVSTAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 10 - protein, 213 aa, Artificial Sequence
  Synthetic Construct
QIVLTQSPAIMSASPGEKVTITCSASSSVSYMHWFQQRPGTSPKLWIYTTSNLASGVPARFSGSGSGTSYSLTISRMEAEDAATYYCQQRSSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 5 aa, Artificial Sequence
  Synthetic Construct
NYWMH

SEQ ID NO: 12 - protein, 17 aa, Artificial Sequence
  Synthetic Construct
YINPSSGYTEYSQKFKD

SEQ ID NO: 13 - protein, 7 aa, Artificial Sequence
  Synthetic Construct
PYGSFAY

SEQ ID NO: 14 - protein, 11 aa, Artificial Sequence
  Synthetic Construct
RASQSISNNLH

SEQ ID NO: 15 - protein, 7 aa, Artificial Sequence
  Synthetic Construct
FASQSIS

SEQ ID NO: 16 - protein, 9 aa, Artificial Sequence
  Synthetic Construct
QQSNSWPYT

SEQ ID NO: 17 - protein, 116 aa, Artificial Sequence
  Synthetic Construct
QVQLQQSGAELAKPGASVRMSCKASGYTFTNYWMHWVKQRPGQGLEWIGYINPSSGYTEYSQKFKDKATLTADKSSSTAYMQLTSLTSEDSAVYYCAGPYGSFAYWGQGTLVTVSA

SEQ ID NO: 18 - protein, 107 aa, Artificial Sequence
  Synthetic Construct
DIKMTQSPATLSVTPGDSVSLSCRASQSISNNLHWYQQKSHESPRLLIKFASQSISGIPSRFSGSGSGTDFTLSINSVETEDFGMYFCQQSNSWPYTFGGGTKLEIK

SEQ ID NO: 19 - protein, 452 aa, Artificial Sequence
  Synthetic Construct
QVQLQQSGAELAKPGASVRMSCKASGYTFTNYWMHWVKQRPGQGLEWIGYINPSSGYTEYSQKFKDKATLTADKSSSTAYMQLTSLTSEDSAVYYCAGPYGSFAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 20 - protein, 214 aa, Artificial Sequence
  Synthetic Construct
DIKMTQSPATLSVTPGDSVSLSCRASQSISNNLHWYQQKSHESPRLLIKFASQSISGIPSRFSGSGSGTDFTLSINSVETEDFGMYFCQQSNSWPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 5 aa, Artificial Sequence
  Synthetic Construct
GYWIE

SEQ ID NO: 22 - protein, 17 aa, Artificial Sequence
  Synthetic Construct
EILPGSGSTNYNEKFKG

SEQ ID NO: 23 - protein, 9 aa, Artificial Sequence
  Synthetic Construct
RTWLYAMDY

SEQ ID NO: 24 - protein, 11 aa, Artificial Sequence
  Synthetic Construct
KASQSVGNNVV

SEQ ID NO: 25 - protein, 7 aa, Artificial Sequence
  Synthetic Construct
YASNRSP

SEQ ID NO: 26 - protein, 8 aa, Artificial Sequence
  Synthetic Construct
QQHYRSFT

SEQ ID NO: 27 - protein, 118 aa, Artificial Sequence
  Synthetic Construct
EVQLKESGAELMKPGASVKLSCKATGYTFTGYWIEWVKQRPGHGLEWIGEILPGSGSTNYNEKFKGKATFTADTSSNTAYMQLSSLTTEDSVIYYCARRTWLYAMDYWGQGTSVTVSS

SEQ ID NO: 28 - protein, 106 aa, Artificial Sequence
  Synthetic Construct
SIVMTQNPKFLPVSAGDRVTMTCKASQSVGNNVVWYQQKPGQSPKLLIYYASNRSPGVPDRFTGSGSGTDFTFTISSVQVEDLAVYFCQQHYRSFTFGTGTKLEIK

SEQ ID NO: 29 - protein, 453 aa, Artificial Sequence
  Synthetic Construct
EVQLKESGAELMKPGASVKLSCKATGYTFTGYWIEWVKQRPGHGLEWIGEILPGSGSTNYNEKFKGKATFTADTSSNTAYMQLSSLTTEDSVIYYCARRTWLYAMDYWGQGTSVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISHSPGK

SEQ ID NO: 30 - protein, 213 aa, Artificial Sequence
  Synthetic Construct
SIVMTQNPKFLPVSAGDRVTMTCKASQSVGNNVVWYQQKPGQSPKLLIYYASNRSPGVPDRFTGSGSGTDFTFTISSVQVEDLAVYFCQQHYRSFTFGTGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 31 - protein, 19 aa, Artificial Sequence
  Synthetic Construct
MEWSWVMLFLLSGTAGVHC

SEQ ID NO: 32 - protein, 22 aa, Artificial Sequence
  Synthetic Construct
MDFQVQIFSFLLISASVIMSRG

SEQ ID NO: 33 - protein, 19 aa, Artificial Sequence
  Synthetic Construct
MKWSWVILFLLSVTAGVHS

SEQ ID NO: 34 - protein, 19 aa, Artificial Sequence
  Synthetic Construct
MGWSCIILFLVATATGVHS

SEQ ID NO: 35 - protein, 19 aa, Artificial Sequence
  Synthetic Construct
MGWSLILLFLVAVATRVLS

SEQ ID NO: 36 - protein, 20 aa, Artificial Sequence
  Synthetic Construct
METQTQVFIFLLLCVSGAHG

SEQ ID NO: 37 - protein, 1 aa, Artificial Sequence
  Synthetic Construct
  VARIANT 1: Can be present in repeats of any integer
G

SEQ ID NO: 38 - protein, 2 aa, Artificial Sequence
  Synthetic Construct
  VARIANT (1)..(2): Can be present in repeats of any integer from 1 to 20
GS

SEQ ID NO: 39 - protein, 5 aa, Artificial Sequence
  Synthetic Construct
  VARIANT (1)..(5): Can be present in repeats of any integer from 1 to 8
GSGGS

SEQ ID NO: 40 - protein, 5 aa, Artificial Sequence
  Synthetic Construct
  VARIANT (1)..(5): Can be present in repeats of any integer from 1 to 8
GGGGS

SEQ ID NO: 41 - protein, 4 aa, Artificial Sequence
  Synthetic Construct
  VARIANT (1)..(4): Can be present in repeats of any integer from 1 to 8
GGGS

SEQ ID NO: 42 - protein, 15 aa, Artificial Sequence
  Synthetic Construct
GGGGSGGGGSGGGGS

SEQ ID NO: 43 - protein, 30 aa, Artificial Sequence
  Synthetic Construct
GGGGSGGGGSGGGGSGGGGSGGGGSGGGGS

SEQ ID NO: 44 - protein, 15 aa, Artificial Sequence
  Synthetic Construct
  VARIANT (1)..(15): Can be present in repeats of any integer from 1 to 3
GSTSGSGKPGSGEGS